(19) United States
(12) Patent Application Publication
(10) Pub. No.: US 2023/0346774 A1
(43) Pub. Date: Nov. 2, 2023

(54) METHODS OF STIMULATING AN ANTI-TUMOR RESPONSE USING A SELECTIVE GLUCOCORTICOID RECEPTOR MODULATOR

(71) Applicant: Corcept Therapeutics Incorporated, Menlo Park, CA (US)

(72) Inventors: Andrew E. Greenstein, Menlo Park, CA (US); Andreas Grauer, Menlo Park, CA (US); Stacie Peacock Shepherd, Menlo Park, CA (US)

(73) Assignee: Corcept Therapeutics Incorporated, Menlo Park, CA (US)

(21) Appl. No.: 17/793,271

(22) PCT Filed: Feb. 10, 2021
(86) PCT No.: PCT/US2021/017259

§ 371 Date: Jul. 15, 2022

Related U.S. Application Data
(60) Provisional application No. 62/972,442, filed on Feb. 10, 2020.

(51) Int. Cl. A61P 35/00; A61K 39/395; A61K 31/4745; A61K 31/337

(52) U.S. Cl. CPC A61K 31/4745; A61K 31/337; A61K 39/3955; A61P 35/00; A61K 2039/505

(57) ABSTRACT

Methods of improving immune function in a cancer patient having a solid tumor are disclosed. The improvement in immune function may slow or stop tumor growth, and may reduce tumor load. Methods include administering effective amounts of a cancer treatment and a nonsteroidal glucocorticoid receptor modulator (GRM) or selective GRM (SGRM). The cancer treatment may include administration of a checkpoint inhibitor. GRM or SGRM administration may induce checkpoint-inhibitor sensitivity in the cancer. Improved immune function may include increased CD8+ T-cell activation, increased pro-inflammatory cytokine secretion, increased TNFα secretion, increased IFNy secretion, and other changes as compared to such activation and secretion prior to GRM administration. In embodiments, immune function is improved after 1, 2, 3, or more days of GRM administration. Other patient characteristics may also be improved by the methods disclosed herein. GRMs include heteroaryl-ketone fused azadecalin and octahydro fused azadecalin GRMs. GRM administration includes oral GRM administration.

### BACKGROUND

Cortisol, an endogenous glucocorticoid receptor (GR) agonist, has broad effects on many bodily systems, including the immune system. Cortisol excess is related to, and causes, many disorders, including Cushing’s syndrome, hyperglycemia, hypertension, hormonal disorders, psychological disorders, and other diseases and disorders. However, cortisol activity is evident even under normal physiological conditions. The normal range for morning serum cortisol, 10-20 ug/dL or 276-552 nM, is in excess of its biochemical K_{D} for the GR ligand binding domain. High morning cortisol prepares the body for the transition from night to day, increasing wakefulness and ensuring immune reactions to foreign agents are moderated. Cortisol action begins by binding to GR. GR binding to cortisol results in agonism of the receptor, trans-repression of cytosolic NFκB signaling, nuclear trafficking, and transactivation of broadly immunosuppressive transcriptional programs.

Glucocorticoid receptor (GR) mediated signaling pathways have dynamic biologic effects involving different components of the immune system and their in vivo effects are unpredictable. For example, glucocorticoids have been reported to have both immunosuppressive effects - such as, suppression of proinflammatory cytokines, promotion of anti-inflammatory cytokines, inhibition of dendritic cells, suppression of natural killer cells, promotion of T-regulatory cells, and induction of T cell apoptosis, - and immune-enhancing effects. See Hinrichs J. Immunother. 2005: 28 (6): 517-524. The effects of GR mediated signaling pathway on cancer cells is likewise elusive. It is believed that activating the GR signaling pathways induce apoptosis in certain types of cancer cells, for example, malignant lymphoid cancers. See Schlossmacher, J. Endocrino. (2011). However, other and contrary effects have also been reported (see, e.g., U.S. Pat. No. 9149485).

Recently, immunotherapy targeting immune checkpoint signaling pathways has been shown to be effective in treating cancer. These pathways suppress immune response and are crucial for maintaining self-tolerance, modulating the duration and amplitude of physiological immune responses in peripheral tissues, and minimizing collateral tissue damage. It is believed that tumor cells can activate the immune checkpoint signaling pathways to decrease the effectiveness of the immune response against tumor tissues. Many of these immune checkpoint signaling pathways are initiated by interactions between checkpoint proteins present on the surface of the cells participating in the immune responses, e.g., T cells, and their ligands, thus they can be readily blocked by agents or modulated by recombinant forms of the checkpoint proteins or ligands or receptors. The agents blocking the immunosuppression pathway induced by checkpoint proteins are commonly referred to as checkpoint inhibitors and a few have been commercialized. Cytotoxic T-lymphocyte-associated antigen 4 (CTLA4, or CTLA-4) antibodies, blocking the immunosuppression pathway by the checkpoint protein CTLA4, were the first of this class of immunotherapeutics to achieve US Food and Drug Administration (FDA) approval. Clinical findings with blockers of additional immune-checkpoint proteins, such as programmed cell death protein 1 (PD-1), indicate broad and diverse opportunities to enhance anti-tumor immunity with the potential to produce durable clinical responses.

GR is expressed in most human cells and is particularly abundant in immune cells. The effects of, and degree of, endogenous cortisol’s effects on the immune system, and their possible consequences for immune responses, including anti-tumor immune responses, are not fully understood. Accordingly, improved methods and treatments for disorders related to cortisol excess, cortisol effects on the immune systems, and for enhancing immune-related treatments are needed.

### SUMMARY

Applicant discloses herein methods of improving immune function in a cancer patient having a solid tumor, comprising administering an effective amount of a cancer treatment and an effective amount of a nonsteroidal glucocorticoid receptor (GR) modulator (GRM), preferably a selective glucocorticoid receptor modulator (SGRM), to said cancer patient, whereby the patient’s immune function is improved. In embodiments, the improvement in immune function is effective to elicit an anti-cancer effect in said patient having a solid tumor, thereby slowing tumor growth, stopping tumor growth, reducing tumor load, or combinations thereof. In embodiments, improved immune function comprises increased CD8+ T-cell activation as compared to CD8+ T-cell activation prior to administration of said nonsteroidal SGRM; improved immune function comprises increased pro-inflammatory cytokine secretion as compared to pro-inflammatory cytokine secretion prior to administration of said nonsteroidal SGRM; improved immune function comprises increased tumor necrosis factor alpha (TNFα) secretion as compared to TNFα secretion prior to administration of said nonsteroidal SGRM; improved immune function comprises increased interferon gamma IFNγ secretion as compared to IFNγ secretion prior to administration of said nonsteroidal SGRM; and combinations thereof. In embodiments, immune function is improved after a few to several days of administration of said nonsteroidal GRM or SGRM (e.g., 1, 2,3, 4, 5, 6, 7, 10, 14, or more days of administration).

In some cases, the GRM (e.g., a SGRM) is a nonsteroidal compound comprising a fused azadecalin structure, wherein the fused azadecalin structure is as described and disclosed in U.S. Pat. 7,928,237 and in U.S. Pat. 8,461,172. In some cases, the GRM (e.g., a SGRM) is a nonsteroidal compound comprising a heteroaryl ketone fused azadecalin structure, wherein the heteroaryl ketone fused azadecalin structure is as described and disclosed in U.S. Pat. 8,859,774. In some cases, the GRM (e.g., a SGRM) is a nonsteroidal compound comprising an octahydro fused azadecalin structure, wherein the octahydro fused azadecalin structure is as described and disclosed in U.S. Pat. 10,047,082.

In some cases, the GRM (e.g., a SGRM, such as a nonsteroidal SGRM) is orally administered.

In embodiments, the GRM is administered with a cancer treatment. In embodiments, the cancer treatment comprises one or more of cancer radiation therapy, administration of growth factor inhibitors, and administration of anti-angiogenesis factors. In embodiments, the cancer treatment comprises administration of a chemotherapeutic agent or an antibody checkpoint inhibitor. In embodiments, the GRM is administered with at least one chemotherapeutic agent. In embodiments, the chemotherapeutic agent is an agent selected from taxanes, alkylating agents, topoisomerase inhibitors, endoplasmic reticulum stress inducing agents, antimetabolites, mitotic inhibitors and combinations thereof. For example, in embodiments, the chemotherapeutic agent is a taxane, such as nab-paclitaxel. In embodiments, the antibody checkpoint inhibitor directed against a protein target selected from PD-1, PD-L1, PD-L2, CTLA-4, LAG3, B7-H3, B7-H4, OX-40, CD137, and TIM3.

To better understand the role of endogenous cortisol in immune suppression, we applied the selective GR antagonist relacorilant to in vitro, in vivo, and ex vivo systems that recapitulate the physiological effects of normal GC activity. These data indicate that antagonizing GR will promote the benefits of ICI therapy. Other improvements and advantages are discussed herein.

### BRIEF DESCRIPTION OF THE DRAWINGS

FIG. 1 shows that glucocorticoid receptor (GR) expression levels (“GR H-score”) correlate with tumor and immune inflitration. CD3+ T-cell infiltration correlated with GR expression in melanoma and TNBC tumors.

FIG. 2 shows that GR expression correlates with PD-L1 expression.

FIG. 3A shows that GR expression positively correlates with CD8+ T-cells and regulatory T-cells (Tregs).

FIG. 3B shows that GR expression negatively correlates with T_{H}1 T-cells and positively correlates with T_{H}2 T-cells.

FIG. 4 shows the restoration of T-cell activation by relacorilant in the presence of physiological levels of cortisol. Expression of CD137 (aka 41-BB) on CD8+ cells was reduced by cortisol and rescued by relacorilant.

FIG. 5 shows, following stimulation by phytohemagglutinin (PHA), suppression of CD3+ cell surface receptors by cortisol, and the restoration of the CD3+ cell surface receptors by relacorilant.

FIG. 6A shows, following stimulation by phytohemagglutinin (PHA), suppression of cytokines and chemokines by cortisol and the restoration of cytokine/chemokine levels by relacorilant. Physiological levels of cortisol suppressed cytokines and chemokines, and this suppression was reversed by relacorilant.

FIG. 6B shows, following stimulation by αCD3 + IL-12, suppression of cytokines and chemokines by cortisol and the restoration of cytokine/chemokine levels by relacorilant. Physiological levels of cortisol suppressed cytokines and chemokines, and this suppression was reversed by relacorilant.

FIG. 7 shows that relacorilant promotes response to an anti-PD1 antagonist antibody (RPM1-14) in the EG7 mouse model. The combination of RMP1-14 and relacorilant was assessed in the EG7 tumor model. Relacorilant significantly increased the efficacy of an anti-PD1 antibody in this model.

FIG. 8 provides further data demonstrating relacorilant’s enhancement of the action of the anti-PD1 antibody in the EG7 model.

FIG. 9 shows the effects of relacorilant alone (group 3) as compared to control (group 1) on serum IL-10 in the EG7 mouse model.

FIG. 10 shows that combined relacorilant + nab paclitaxel treatment suppressed gene expression in patients with solid tumors. Suppressed genes included IL8 (CXCL8), IDO1, and EP4 (PTGER4) (n=46).

FIG. 11 shows a summary of effects on selected biomarkers in a patient with complete response (CR) to treatment with relacorilant + nab-paclitaxel. This patient exhibited a decrease in neutrophil-to-lymphocyte ratio (NLR), and changes in CD4+ cells, CD8+ cells, CD3+ T-cells, expression of ptgs2 and dusp1m and other changes. (C1D1 indicates cycle 1 day 1 of treatment; C1D15 indicates cycle 1 day 15 of treatment; C4D1 indicates cycle 4 day 1 of treatment, and EOT indicates end of treatment.)

FIG. 12 provides a table summarizing characteristics and prior treatments of human cancer patients who responded well to the combined relacorilant + nab-paclitaxel treatment. (PR indicates partial response; CR indicates complete response; SD indicates stable disease (no tumor progression).)

FIG. 13 further illustrates effects on NLR, transcription of GR-controlled genes, immunomodulatory cytokines, and immune cells in human cancer patients who responded extemely well to the combined relacorilant + nab-paclitaxel treatment.

FIG. 14 illustrates the effects of short-term relacorilant treatment on T-cell function. The results of a short term pharmacodynamic study (conducted to assess the effects of relacorilant on T-cell function prior to any observer able effects on tumor volume) show that mean body weight and tumor volume were unaffected by any treatment assessed during this timeframe.

FIG. 15 illustrates the short-term effects of GR antagonism in combination with α PD1 in the EG7 syngeneic model. In a 7-day pharmacodynamic study, relacorilant + αPD1 increased antigen specific T-cells in the spleen (left) and tumor (right).

FIG. 16 illustrates the effects of relacorilant and αPD1 on spleen cells assessed after a 7-day EG7 study. PD1 expression (top left) and CD69 expression (top right) in splenic CD8+ T-cells are shown as a percentage of CD8+ T-cells. CD3+CD8+ T-cells are shown as a percent of splenic CD45.1+ cells (bottom left). P values from unpaired non-parametric T-tests are shown.

FIG. 17 illustrates the effects of relacorilant and αPD1, TNFα, and IL-6 levels in serum assessed after a 7 day EG7 study.

### DETAILED DESCRIPTION

### A. Introduction

GR expression was observed in human tumor and immune cells, and its abundance was positively correlated with PDL1 expression and tumor infiltration of Th2 and Treg cells while negatively correlated with Th1 cell infiltration. Cortisol inhibited, and relacorilant restored, T-cell activation and pro-inflammatory cytokine secretion in human PBMC’s stimulated in vitro. In the EG7 mouse model, relacorilant significantly increased the efficacy of an anti-PD1 antibody. In a phase I nab-paclitaxel combination study in patients with advance solid tumors, relacorilant suppressed the expression IL-8, EP4, and IDO1 systemically and normalized the neutrophil-to-lymphocyte ratio (NLR). In a subset of patients with sustained response, relacorilant increased CD3+ cells and IFNγ, decreased Tregs and IL-10, and suppressed transcription of known GR-controlled genes. Together, these data characterize the broad immunosuppressive effects of cortisol that can be reversed by relacorilant.

Applicant discloses herein the effects of selective glucocorticoid receptor modulators (SGRMs). Many SGRMs are GR antagonists. For example, relacorilant is a potent and selective GR antagonist. Half-maximal GR binding was observed at 0.15 nM while progesterone receptor (PR) binding was not observed at concentrations in excess of 1000 nM. In human stimulated PBMCs, TNF-α is suppressed by GR agonists and relacorilant restored TNF-α production with half maximal effect observed at 9 nM. Relacorilant, administered orally at doses that achieved systemic exposure similar to those seen in phase I studies, normalized glucose and insulin in a rat model of corticosterone-induced insulin resistance. Phase I healthy volunteer studies demonstrated tolerability and the ability to reverse the pharmacodynamic effects of a single dose of prednisone. GR agonist pharmacodynamic effects included the induction of FKBP5 mRNA, a canonical GR-controlled gene, in whole blood and the suppression of eosinophil abundance in whole blood, both of which were reversed by relacorilant. Unlike mifepristone, a steroid analog and hormone receptor modulator, GR inverse agonism was not observed with relacorilant. In a Phase II study in patients with Cushing’s disease, relacorilant demonstrated the ability to reverse the effects of excess cortisol on hypertension and insulin resistance.

Applicant discloses herein methods of improving immune function in a cancer patient having a solid tumor, comprising administering an effective amount of a cancer treatment and an effective amount of a nonsteroidal selective glucocorticoid receptor modulator (SGRM) to said cancer patient, whereby the patient’s immune function is improved. Such improved immune function may include improvement in the patient’s immune system to elicit an anticancer effect. In embodiments, the improvement in immune function is effective to elicit an anti-cancer effect in said patient having a solid tumor, thereby slowing tumor growth, stopping tumor growth, reducing tumor load, or combinations thereof. In embodiments, improved immune function comprises increased CD8+ T-cell activation as compared to CD8+ T-cell activation prior to administration of said nonsteroidal SGRM; improved immune function comprises increased pro-inflammatory cytokine secretion as compared to pro-inflammatory cytokine secretion prior to administration of said nonsteroidal SGRM; improved immune function comprises increased TNFα secretion as compared to TNFα secretion prior to administration of said nonsteroidal SGRM; improved immune function comprises increased IFNγ secretion as compared to IFNγ secretion prior to administration of said nonsteroidal SGRM; and combinations thereof. In embodiments, immune function is improved after a few to several days of administration of said nonsteroidal GRM or SGRM (e.g., 1, 2,3, 4, 5, 6, 7, 10, 14, or more days of administration).

In embodiments of the methods disclosed herein, the nonsteroidal SGRM is a compound comprising a heteroaryl ketone fused azadecalin structure having the formula:

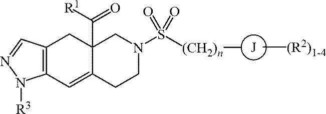

wherein
R¹ is a heteroaryl ring having from 5 to 6 ring members and from 1 to 4 heteroatoms each independently selected from the group consisting of N, O and S, optionally substituted with 1-4 groups each independently selected from R^{1a},
each R^{1a} is independently selected from the group consisting of hydrogen, C₁₋₆ alkyl, halogen, C₁₋₆ haloalkyl, C₁₋₆ alkoxy, C₁₋₆ haloalkoxy, CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C₁₆ haloalkyl, C₁₆ alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl, wherein the heterocycloalkyl groups are optionally substituted with 1-4 R^{2c} groups;
alternatively, two R² groups linked to the same carbon are combined to form an oxo group (=O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, CN, and NR^{2a}R^{2b},
each R^{2d} is independently selected from the group consisting of hydrogen and C₁₋₆ alkyl, or two R^{2d} groups attached to the same ring atom are combined to form (=O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl; and
subscript n is an integer from 0 to 3;
or salts and isomers thereof.

In embodiments of the methods where the nonsteroidal SGRM is a heteroaryl-ketone fused azadecalin, the nonsteroidal SGRM is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, termed relacorilant, which has the following structure:

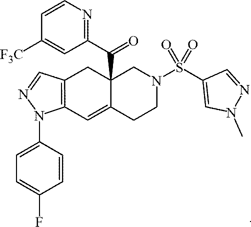

In embodiments of the methods where the nonsteroidal selective GRA is a heteroaryl-ketone fused azadecalin, the nonsteroidal SGRM is (R)-(1-(4-fluorophenyl)-6-((4-(trifluoromethyl)phenyl)sulfonyl)-4,4a,5,6,- 7,8-hexahydro-1H-pyrazolo[3,4-g]isoquinolin-4a-yl)(thiazol-2-yl)methanone, termed CORT122928, which has the following structure:

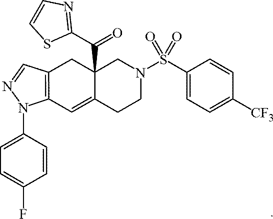

In embodiments of the methods where the nonsteroidal SGRM comprises a heteroaryl-ketone fused azadecalin, the nonsteroidal SGRM is (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone, termed CORT113176, which has the following structure:

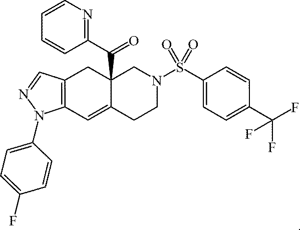

In embodiments of the methods disclosed herein, the nonsteroidal SGRM comprises an octahydro fused azadecalin structure compound having the formula:

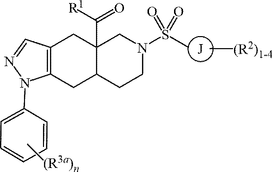

wherein
R¹ is a heteroaryl ring having from 5 to 6 ring members and from 1 to 4 heteroatoms each independently selected from the group consisting of N, O and S, optionally substituted with 1-4 groups each independently selected from R^{1a},
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl;
ring J is selected from the group consisting of an aryl ring and a heteroaryl ring having from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, -NR^{2a}R^{2b}, -C(O)R^{2a}, -C(O)OR^{2a}, -C(O)NR^{2a}R^{2b}, -SR^{2a}, -S(O)R^{2a}, -S( O)₂R^{2a}, C₃₋₈ cycloalkyl, and C₃₋₈ heterocycloalkyl having from 1 to 3 heteroatoms each independently selected from the group consisting of N, O and S;
alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups;
R^{2a}, R^{2b} and R^{2c} are each independently selected from the group consisting of hydrogen and C₁₋₆ alkyl;
each R^{3a} is independently halogen; and
subscript n is an integer from 0 to 3;
or salts and isomers thereof.

In embodiments of the methods disclosed herein, the nonsteroidal SGRM comprises an octahydro fused azadecalin structure compound having the formula:

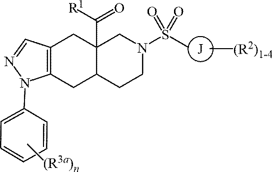

Wherein R¹ is selected from the group consisting of pyridine and thiazole, optionally substituted with 1-4 groups each independently selected from R^{1a},
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl; ring J is selected from the group consisting of phenyl, pyridine, pyrazole, and triazole;
each R² is independently selected from the group consisting of hydrogen, C₁₋₆ alkyl, halogen, C₁₋₆ haloalkyl, and —CN;
R^{3a} is F;
subscript n is an integer from 0 to 3,
or salts and isomers thereof.

In embodiments where the nonsteroidal SGRM comprises an octahydro fused azadecalin structure, the nonsteroidal SGRM is ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone, termed exicorilant, which has the structure:

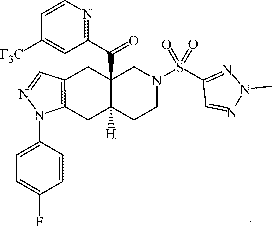

In embodiments, the nonsteroidal SGRM is the octahydro fused azadecalin compound having the chemical name ((4aR,8aS)-1-(4-fluorophenyl)-6-((2-isopropyl-2H-1,2,3-triazol-4-yl)sulfonyl)-4,4a,5,6,7,8,8a,9-octahydro-1H-pyrazolo[3,4-g]isoquinolin-4a-yl)(thiazol-2-yl)methanone, termed “CORT125329”, having the formula:

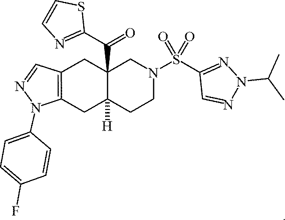

In some cases, the effective amount of the GRM (e.g., a SGRM, such as a nonsteroidal SGRM) is a daily dose of between 1 and 100 mg/kg/day, or between about 1 and 20 mg/kg/day. In some embodiments, the daily dose of the GRM is 1, 2, 4, 6, 8, 10, 12, 14, 16, 18, 20, 30, 40, 50 60, 70, 80, 90 or 100 mg/kg/day. In some cases, the GRM is administrated for at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or 80 weeks. In embodiments, the GRM is a SGRM. In preferred embodiments, the GRM is a GR antagonist (a GRA), and may be a selective GRA.

In embodiments, the GRM is administered with a cancer treatment. In embodiments of the methods disclosed herein, the cancer treatment comprises administration of a chemotherapeutic agent. In embodiments, the chemotherapeutic agent is selected from the group consisting of taxanes, alkylating agents, topoisomerase inhibitors, endoplasmic reticulum stress inducing agents, antimetabolites, mitotic inhibitors and combinations thereof.

In embodiments, the chemotherapeutic agent is a taxane, and may be, e.g., nab-paclitaxel.

In embodiments of the methods disclosed herein, the cancer treatment comprises administration of an immunotherapeutic agent. For example, in embodiments of the methods disclosed herein, the cancer treatment includes administration of an antibody checkpoint inhibitor. Thus, in embodiments, the methods disclosed herein comprise administration of an antibody checkpoint inhibitor (an antibody directed against a protein target) that is directed to a target selected from PD-1, PD-L1, PD-L2, CTLA-4, LAG3, B7-H3, B7-H4, OX-40, CD137, and TIM3. In embodiments, the cancer treatment comprises one or more of cancer radiation therapy, administration of growth factor inhibitors, and administration of anti-angiogenesis factors.

In embodiments of the methods disclosed herein, the cancer treatment comprises a method of treating a subject suffering from a solid tumor, comprising identifying a patient suffering from a solid tumor and having excess cortisol; administering a combination treatment comprising administration of 1) a selective glucocorticoid receptor modulator (SGRM) and 2) a cancer chemotherapy agent; thereby restoring CD8+ T-cell activation, restoring pro-inflammatory cytokine secretion, or both. In embodiments, the methods include one of more of increasing T-cell numbers, increasing plasma interferon γ (IFNγ), decreasing Treg cells, decreasing interleukin-10 (IL-10) and combinations thereof.

### Definitions

As used herein, the genes cxcl8, idol, and ptger4 and others refer to the following:

| | | | |
|---|---|---|---|
| Gene | Accession | HUGO symbol | Nanostring ID |
| IL-6 | NM_000600.3 | IL6 | NM_000600.3:364 |
| IL-8 | NM_000584.3 | CXCL8 | NM_000584.3:170 |
| IL-10 | NM_000572.2 | IL10 | NM_000572.2:230 |
| IDO1 | NM_002164.5 | IDO1 | NM_002164.5:52 |
| EP4 | NM_000958.2 | PTGER4 | NM_000958.2:975 |
| DUSP1 | NM_004417.2 | DUSP1 | NM_004417.2:987 |
| COX2 | NM_000963.3 | PTGS2 | NM_000963.3:450 |

As used herein, the term “tumor” and the term “cancer” are used interchangeably and both refer to an abnormal growth of tissue that results from excessive cell division. A tumor that invades the surrounding tissue and/or can metastasize is referred to as “malignant.” A tumor that does not metastasize is referred to as “benign.”

As used herein, the term “patient” refers to a human that is or will be receiving, or has received, medical care for a disease or condition.

As used herein, the terms “administer,” “administering,” “administered” or “administration” refer to providing a compound or a composition (e.g., one described herein), to a subject or patient. For example, a compound or composition may be administered orally to a patient.

As used herein, the term “effective amount” or “therapeutic amount” refers to an amount of a pharmacological agent effective to treat, eliminate, or mitigate at least one symptom of the disease being treated. In some cases, “therapeutically effective amount” or “effective amount” can refer to an amount of a functional agent or of a pharmaceutical composition useful for exhibiting a detectable therapeutic or inhibitory effect. The effect can be detected by any assay method known in the art. The effective amount can be an amount effective to invoke an antitumor response. For the purpose of this disclosure, the effective amount of SGRM or the effective amount of a chemotherapeutic agent is an amount that would reduce tumor load or bring about other desired beneficial clinical outcomes related to cancer improvement when combined with a chemotherapeutic agent or SGRM, respectively.

As used herein, the terms “administer,” “administering,” “administered” or “administration” refer to providing a compound or a composition (e.g., one described herein), to a subject or patient. Administration may be by oral administration (i.e., the subject receives the compound or composition via the mouth, as a pill, capsule, liquid, or in other form suitable for administration via the mouth. Oral administration may be buccal (where the compound or composition is held in the mouth, e.g., under the tongue, and absorbed there). Administration may be by injection, i.e., delivery of the compound or composition via a needle, microneedle, pressure injector, or other means of puncturing the skin or forcefully passing the compound or composition through the skin of the subject. Injection may be intravenous (i.e., into a vein); intraarterial (i.e., into an artery); intraperitoneal (i.e., into the peritoneum); intramusucular (i.e., into a muscle); or by other route of injection. Routes of administration may also include rectal, vaginal, transdermal, via the lungs (e.g., by inhalation), subcutaneous (e.g., by absorption into the skin from an implant containing the compound or composition), or by other route.

As used herein, the term “combination therapy” refers to the administration of at least two pharmaceutical agents to a subject to treat a disease. The two agents may be administered simultaneously, or sequentially in any order during the entire or portions of the treatment period. The at least two agents may be administered following the same or different dosing regimens. In some cases, one agent is administered following a scheduled regimen while the other agent is administered intermittently. In some cases, both agents are administered intermittently. In some embodiments, the one pharmaceutical agent, e.g., a SGRM, is administered daily, and the other pharmaceutical agent, e.g., a chemotherapeutic agent, is administered every two, three, or four days.

As used herein, the term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless of whether it exists in a free form or an associated form.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

As used herein, the term “Adrenocorticotrophic Hormone” (ACTH) refers to the peptide hormone produced and secreted by the anterior pituitary gland that stimulates the adrenal cortex to secrete glucocorticoid hormones, which help cells synthesize glucose, catabolize proteins, mobilize free fatty acids and inhibit inflammation in allergic responses. One such glucocorticoid hormone is cortisol, which regulates metabolism of carbohydrate, fat, and protein metabolism. In healthy mammals, ACTH secretion is tightly regulated. ACTH secretion is positively regulated by corticotropin releasing hormone (CRH), which is released by the hypothalamus. ACTH secretion is negatively regulated by cortisol and other glucocorticoids.

The terms “adrenal hormone”, “adrenal pre-hormone”, and “adrenal hormone or adrenal pre-hormone” refer to steroid molecules that are, or are precursors of, hormones produced by the adrenal gland. As used herein, without limitation, an “adrenal hormone or adrenal pre-hormone” may be one or more of 17α-hydroxy pregnenolone, 17α-hydroxy progesterone, 11-deoxycortisol, pregnenolone, progesterone, 11-deoxycorticosterone, corticosterone, 18-hydroxycorticosterone, aldosterone, dehydroepiandrosterone (androstenolone, DHEA), dehydroepiandrosterone sulfate (DHEA-S), and androstenedione. As used herein, the terms “adrenal hormone”, “adrenal pre-hormone”, and “adrenal hormone or adrenal pre-hormone” refer to hormones and pre-hormones other than cortisol unless it is explicitly stated that cortisol in intended to be included as well.

The term “measuring the level,” in the context of ACTH, cortisol, adrenal hormone, adrenal pre-hormone, or other hormone or other steroid, refers determining, detecting, or quantitating the amount, level, or concentration of, for example, cortisol, ACTH or other steroid in a sample obtained from a subject. The sample may be, e.g., a blood sample, a saliva sample, a urine sample, or other sample obtained from the patient. A level may be measured from a fraction of a sample. For example, a level (e.g., ACTH or cortisol) may be measured in the plasma fraction of a blood sample; may be measured in a serum fraction of a blood sample; or, in embodiments, may be measured in whole blood.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Cells of the immune system are identified herein according to the commonly used and commonly accepted terminology in the art. For example, the terms “Treg” and “T_{reg}” are used interchangeably herein to refer to regulatory T-cells. “IFN” refers to an interferon, so that, for example, IFNγ refers to interferon gamma. “IL” refers to an interleukin, so that, for example, IL-10 refers to interleukin 10. “TNF” refers to tumor necrosis factor, so that, for example, TNFα refers to tumor necrosis factor alpha. Other terms and acronyms are known and used by those of ordinary skill in the art.

As used herein, the term “checkpoint-inhibitor-sensitive cancer” refers to a cancer that is responsive to checkpoint inhibitors. Administration of one or more checkpoint inhibitors to patients having such a tumor would cause a reduction in the tumor load or other desired beneficial clinical outcome related to cancer improvement.

As used herein, the phrase “an amount effective to potentiate” refers to the amount of of a pharmacological agent effective to enhance the activity of another therapeutic agent in treating, eliminating, or mitigating at least one symptom of the disease being treated. The agent used to potentiate the activity of another can be effective or non-effective in treating, eliminating, or mitigating the symptom of the disease itself. In some cases, the potentiating agent is not effective, and the effect of potentiation can be shown by the increased degree in relieving the symptom resulting from treatment by the combination of the two agents as compared to the treatment with the therapeutic agent alone. In some cases, the potentiating agent itself is effective in treating the symptoms, and the potentiating effect can be shown by a synergistic effect between the potentiating agent and the therapeutic agent. For example, a SGRM may act as a potentiating agent to potentiate the activity of checkpoint inhibitors in treating cancer, regardless whether the SGRM would be effective in treating the cancer if administered alone. In some embodiments, a potentiating effect of 10% to 1000% can be achieved. In some embodiments, the SGRM is administered at an amount that renders the tumor sensitive to the checkpoint inhibitor, i.e., a showing of a reduction of tumor load or other related clinical benefit that would not otherwise appear when the tumor is treated with the checkpoint inhibitor in the absence of the SGRM.

As used herein, the term “checkpoint protein” refers to a protein that is present on the surface of certain types of cells, e.g. T cells and certain tumor cells, and can induce checkpoint signaling pathways and result in suppression of immune responses. Commonly known checkpoint proteins include CTLA4, PD-1, PD-L1, PD-L2, LAG3, B7-H3, B7-H4, TIM3, CD160, CD244, VISTA, TIGIT, and BTLA. (Pardoll, 2012, Nature Reviews Cancer 12:252-264; Baksh, 2015, Semin Oncol. 2015 Jun;42(3):363-77). For example, CTLA4, PD-1 and PD-L1 are well studied and therapies targeting these proteins are well-used clinical therapies.

In some cases, the checkpoint inhibitor is a small molecule, non-protein compound that inhibits at least one checkpoint protein. In one embodiment, the checkpoint inhibitor is a small molecule, non-protein compound that inhibits a checkpoint protein selected from the group consisting of CTLA-4, PD-1, PD-L1, PD-L2, LAG3, B7-H3, B7-H4, TIM3, CD160, CD244, VISTA, TIGIT, and BTLA.

In some cases, the checkpoint inhibitor is an antibody against at least one checkpoint protein, e.g., PD-1, CTLA-4, PD-L1, PD-L2, CTLA-4, LAG3, B7-H3, B7-H4, TIM3, CD160, CD244, VISTA, TIGIT, and BTLA. In some cases, the checkpoint inhibitor is an antibody that is effective against two or more of the checkpoint proteins selected from the group of PD-1, CTLA-4, PD-L1, PD-L2, AG3, B7-H3, B7-H4, TIM3, CD160, CD244, VISTA, TIGIT, and BTLA.

In some cases, the checkpoint inhibitor is an antibody targeted against a checkpoint protein, or against more than one checkpoint protein. Such antibody checkpoint inhibitors may be termed “α” and identified by preceding the name of the target protein by the Greek letter “α”. Thus, an antibody checkpoint inhibitor directed against PD1 may be termed “αPD1”, an antibody checkpoint inhibitor directed against CD3 may be termed “αCD3”, and so forth. Treatments involving administration of such antibody checkpoint inhibitors may also be identified in the same way, so that a treatment using an anti-PD1 antibody may be termed “αPD1” or an “αPD1 treatment”, a treatment using an anti-CD3 antibody may be termed “αCD3” or an “αCD3 treatment”, and so forth.

As used herein, the term “PD-1” refers to Programmed Cell Death Protein 1 (also known as CD279), a cell surface membrane protein of the immunoglobulin superfamily. PD-1 is expressed by B cells, T cells and NK cells. The major role of PD-1 is to limit the activity of T cells in peripheral tissues during inflammation in response to infection, as well as to limit autoimmunity. PD-1 expression is induced on activated T cells and binding of PD-1 to one of its endogenous ligands acts to inhibit T cell activation by inhibiting stimulatory kinases. PD-1 also acts to inhibit the TCR “stop signal”. PD-1 is highly expressed on Treg cells (regulatory T cells) and may increase their proliferation in the presence of ligand (Pardoll, 2012, Nature Reviews Cancer 12:252-264).

As used herein, the term “PD-L1” refers to Programmed Cell Death ligand 1 (also known as CD274 and B7-H1), a ligand for PD-1. PD-L1 is found on activated T cells, B cells, myeloid cells, macrophages, and tumor cells. Although there are two endogenous ligands for PD-1, PD-L1 and PD-L2, anti-tumor therapies have focused on anti-PD-L1. The complex of PD-1 and PD-L1 inhibits proliferation of CD8+ T cells and reduces the immune response (Topalian et al., 2012, N. Engl J. Med. 366:2443-54; Brahmer et al., 2012, N. Engl J. Med. 366:2455-65).

As used herein, the term “PD-L2” refers to Programmed Cell Death ligand 2. PD-L2 competes with PD-L1 for binding to PD-1.

As used herein, the terms “CTLA4” and “CTLA-4” refer to Cytotoxic T-lymphocyte antigen 4 (also known as CD152), a member of the immunoglobulin superfamily that is expressed exclusively on T cells. CTLA4 acts to inhibit T cell activation and is reported to inhibit helper T cell activity and enhance regulatory T cell immunosuppressive activity. Although the precise mechanism of action of CTL4-A remains under investigation, it has been suggested that it inhibits T cell activation by outcompeting CD28 in binding to CD80 and CD86 on antigen presenting cells, as well as actively delivering inhibitor signals to the T cell (Pardoll, 2012, Nature Reviews Cancer 12:252-264).

As used herein, the term “LAG3” refers to Lymphocyte Activation Gene-3 (also termed CD223).

As used herein, the term “B7-H3” refers to the immune checkpoint protein also known as CD276; B7-H3 is often overexpressed on cancer cells (e.g., some solid tumors).

As used herein, the term “B7-H4” refers to the immune checkpoint protein also known as V-set domain-containing T-cell activation inhibitor 1, which may be present on the surface of antigen-presenting cells.

As used herein, the term “TIM3” refers to the protein also known as T cell immunoglobulin and mucin domain-containing protein 3.

As used herein, the term “CD160” refers to the 27 kiloDalton glycoprotein encoded by the CD160 gene in humans. The expression of CD160 is tightly associated with peripheral blood NK cells and CD8 T lymphocytes with cytolytic effector activity.

As used herein, the term “CD244” refers to the protein also known as “Cluster of Differentiation 244”. It is a member of the immunoregulatory receptor Signaling Lymphocyte Activation Molecule (SLAM) family.

As used herein, the term “VISTA” refers to immune checkpoint protein also known as V-domain Ig suppressor of T cell activation. It is encoded by the C10orf54 gene.

As used herein, the term “TIGIT” (T cell immunoreceptor with Ig and ITIM domains) refers to the immune receptor protein also called WUCAM and Vstm3.

As used herein, the term “BTLA” (B- and T-lymphocyte attenuator) refers to the checkpoint protein encoded in humans by the BTLA gene. It is also termed CD272 (cluster of differentiation 272).

As used herein, the term “checkpoint inhibitor” refers to any molecule, including antibodies and small molecules, that blocks the immunosuppression pathway induced by one or more checkpoint proteins.

As used herein, the term “antibody” as used herein also includes a full-length antibody as well as an “antigen-binding portion” of an antibody. The term “antigen-binding portion”, as used herein, refers to one or more fragments of an antibody that retain the ability to specifically bind to an antigen (e.g., PD-1). Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG molecules or other isotypes. VH and VI can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Antibodies may be polyclonal or monoclonal; xenogeneic, allogeneic, or syngeneic; or modified forms thereof, e.g. humanized, chimeric, etc. Antibodies of the invention bind specifically or substantially specifically to one or more checkpoint proteins. The term “monoclonal antibodies” refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition typically displays a single binding affinity for a particular antigen with which it immunoreacts.

As used herein, the term “antibody effective against a checkpoint protein” refers to an antibody that can bind to the checkpoint protein and antagonize the checkpoint protein’s function in suppressing immune response. For example, an antibody against PD-1 refers to an antibody that can bind to PD-1 and block the PD-1′s inhibitory function on the immune response, through e.g., blocking the interactions between PD-1 and PD-L1. In some cases, an antibody can be against two checkpoint proteins, i.e., having the ability of binding to two checkpoint proteins and inhibiting their function.

The term “cortisol” refers to the naturally occurring glucocorticoid hormone (also known as hydrocortisone) that is produced by the zona fasciculata of the adrenal gland. Cortisol has the structure:

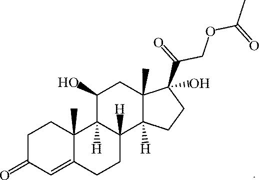

The term “total cortisol” refers to cortisol that is bound to cortisol-binding globulin (CBG or transcortin) and free cortisol (cortisol that is not bound to CBG). The term “free cortisol” refers to cortisol that is not bound to cortisol-binding globulin (CBG or transcortin). As used herein, the term “cortisol” refers to total cortisol, free cortisol, and/or cortisol bound of CBG.

The term “glucocorticosteroid” (“GC”) or “glucocorticoid” refers to a steroid hormone that binds to a glucocorticoid receptor. Glucocorticosteroids are typically characterized by having 21 carbon atoms, an α,β-unsaturated ketone in ring A, and an α-ketol group attached to ring D. They differ in the extent of oxygenation or hydroxylation at C-11, C-17, and C-19; see Rawn, “Biosynthesis and Transport of Membrane Lipids and Formation of Cholesterol Derivatives,” in Biochemistry, Daisy et al. (eds.), 1989, pg. 567.

As used herein, the phrase “not otherwise indicated for treatment with a glucocorticoid receptor modulator” refers to refers to a patient that is not suffering from any condition recognized by the medical community to be effectively treatable with glucocorticoid receptor antagonists, with the exception of hepatic steatosis. Conditions known in the art and accepted by the medical community to be effectively treatable with glucocorticoid receptor antagonists include: psychosis associated with interferon-α therapy, psychotic major depression, dementia, stress disorders, autoimmune disease, neural injuries, and Cushing’s syndrome.

A mineralocorticoid receptor (MR), also known as a type I glucocorticoid receptor (GR I), is activated by aldosterone in humans.

As used herein, the term “glucocorticoid receptor” (“GR”) refers to the type II GR, a family of intracellular receptors which specifically bind to cortisol and/or cortisol analogs such as dexamethasone (See, e.g., Turner & Muller, J. Mol. Endocrinol. Oct. 1, 2005 35 283-292). The glucocorticoid receptor is also referred to as the cortisol receptor. The term includes isoforms of GR, recombinant GR and mutated GR.

The term “glucocorticoid receptor modulator” (GRM) refers to any compound which modulates GC binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, decreases the activity of tyrosine aminotransferase (TAT) in HepG2 cells. TAT activity can be measured as outlined in the literature by A. Ali et al., J. Med. Chem., 2004, 47, 2441-2452.

As used herein, the term “selective glucocorticoid receptor modulator” (SGRM) refers to any composition or compound which modulates GC binding to GR, or modulates any biological response associated with the binding of a GR to an agonist. By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor modulator bind GR with an affinity that is 10× greater (⅒^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor modulator binds GR with an affinity that is 100× greater (1/100^{th} the K_{d} value) than its affinity to the MR, AR, or PR, both the MR and PR, both the MR and AR, both the AR and PR, or to the MR, AR, and PR. In another embodiment, the selective glucocorticoid receptor modulator binds GR with an affinity that is 1000× greater (1/1000^{th} the K_{d} value) than its affinity to the MR, AR, or PR, both the MR and PR, both the MR and AR, both the AR and PR, or to the MR, AR, and PR. Relacorilant is a SGRM.

“Glucocorticoid receptor antagonist” (GRA) refers to any compound which inhibits GC binding to GR, or which inhibits any biological response associated with the binding of GR to an agonist. Accordingly, GR antagonists can be identified by measuring the ability of a compound to inhibit the effect of dexamethasone. TAT activity can be measured as outlined in the literature by A. Ali et al., J. Med. Chem., 2004, 47, 2441-2452. A GRA is a compound with an IC₅₀ (half maximal inhibition concentration) of less than 10 micromolar. See Example 1 of U.S. Pat. 8,859,774, the entire contents of which is hereby incorporated by reference in its entirety.

As used herein, the term “selective glucocorticoid receptor antagonist” (SGRA) refers to any composition or compound which inhibits GC binding to GR, or which inhibits any biological response associated with the binding of a GR to an agonist (where inhibition is determined with respect to the response in the absence of the compound). By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor antagonist bind GR with an affinity that is 10x greater (⅒^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor antagonist binds GR with an affinity that is 100x greater (1/100^{th} the K_{d} value) than its affinity to the MR, AR, or PR, both the MR and PR, both the MR and AR, both the AR and PR, or to the MR, AR, and PR. In another embodiment, the selective glucocorticoid receptor antagonist binds GR with an affinity that is 1000x greater (1/1000^{th} the K_{d} value) than its affinity to the MR, AR, or PR, both the MR and PR, both the MR and AR, both the AR and PR, or to the MR, AR, and PR. Relacorilant is a SGRA.

Nonsteroidal GRA, SGRA, GRM, and SGRM compounds include compounds comprising a fused azadecalin structure (which may also be termed a fused azadecalin backbone), compounds comprising a heteroaryl-ketone fused azadecalin structure (which may also be termed a heteroaryl-ketone fused azadecalin backbone), and compounds comprising an octahydro fused azadecalin structure (which may also be termed an octahydro fused azadecalin backbone).

Exemplary nonsteroidal GRA, SGRA, GRM, and SGRM compounds comprising a fused azadecalin structure include those described in U.S. Pat. Nos. 7,928,237 and 8,461,172. Exemplary nonsteroidal GRA, SGRA, GRM, and SGRM compounds comprising a heteroaryl-ketone fused azadecalin structure include those described in U.S. Pat. 8,859,774. Exemplary nonsteroidal GRA, SGRA, GRM, and SGRM compounds comprising an octahydro fused azadecalin structure include those described in U.S. Pat. 10,047,082. All patents, patent publications, and patent applications disclosed herein are hereby incorporated by reference in their entireties.

Exemplary glucocorticoid receptor antagonists comprising a fused azadecalin structure include those described in U.S. Pat. No. 7,928,237; and U.S. Pat. No. 8,461,172. In embodiments, the fused azadecalin GRA is the compound (R)-4-a-ethoxymethyl-1-(4-fluoro-phenyl)-6-(4-trifluoromethyl-benzenesulfonyl)-4,4a,5,6,7,8-hexahydro-1H,1,2,6-triaza-cyclopenta[b]naphthalene (“CORT108297”), which has the structure:

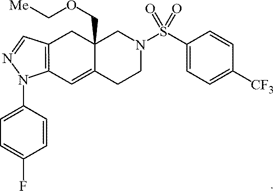

Exemplary heteroaryl-ketone fused azadecalin compounds are described in U.S. Pat. 8,859,774; in U.S. Pat. 9,273,047; in U.S. Pat. 9,707,223; and in U.S. Pat. 9,956,216, all of which patents are hereby incorporated by reference in their entireties. In embodiments, the heteroaryl-ketone fused azadecalin GRA is the compound (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone (Example 18 of U.S. 8,859,774), also known as “relacorilant” and as “CORT125134”, which has the following structure:

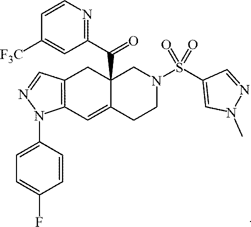

In embodiments, the heteroaryl-ketone fused azadecalin GRA is the compound (R)-(1-(4-fluorophenyl)-6-((4-(trifluoromethyl)phenyl)sulfonyl)-4,4a,5,6,- 7,8-hexahydro-1H-pyrazolo[3,4-g]isoquinolin-4a-yl)(thiazol-2-yl)methanone (termed “CORT122928”), which has the following structure:

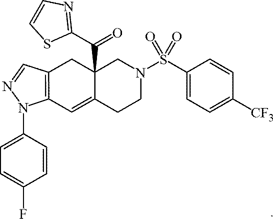

In embodiments, the heteroaryl-ketone fused azadecalin GRA is the compound (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone (termed “CORT113176”), which has the following structure:

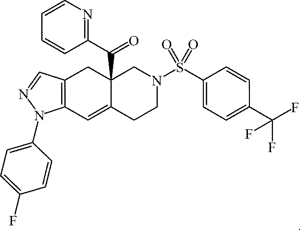

Exemplary glucocorticoid receptor antagonists comprising an octohydro fused azadecalin structure include those described in U.S. Pat. No. 10,047,082. In embodiments, the octahydro fused azadecalin compound is the compound ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone (termed exicorilant, or CORT125281) which has the structure:

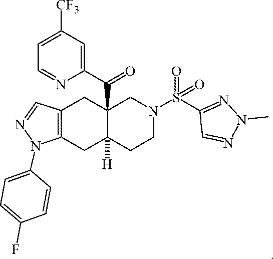

In some cases, the nonsteroidal SGRM is CORT125329, i.e., ((4aR,8aS)-1-(4-fluorophenyl)-6-((2-isopropyl-2H-1,2,3-triazol-4-yl)sulfonyl)-4,4a,5,6,7,8,8a,9-octahydro-1H-pyrazolo[3,4-g]isoquinolin-4a-yl)(thiazol-2-yl)methanone, which has the following structure:

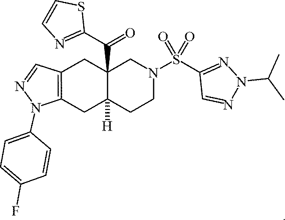

As used herein, the term “composition” is intended to encompass a product comprising the specified ingredients such as the said compounds, their tautomeric forms, their derivatives, their analogues, their stereoisomers, their polymorphs, their deuterated species, their pharmaceutically acceptable salts, esters, ethers, metabolites, mixtures of isomers, their pharmaceutically acceptable solvates and pharmaceutically acceptable compositions in specified amounts, as well as any product which results, directly or indirectly, from combination of the specified ingredients in the specified amounts. Such term in relation to a pharmaceutical composition is intended to encompass a product comprising the active ingredient (s), and the inert ingredient (s) that make up the carrier, as well as any product which results, directly or indirectly, in combination, complexation or aggregation of any two or more of the ingredients, or from dissociation of one or more of the ingredients, or from other types of reactions or interactions of one or more of the ingredients. Accordingly, the pharmaceutical compositions of the present invention are meant to encompass any composition made by admixing compounds of the present invention and their pharmaceutically acceptable carriers.

In some embodiments, the term “consisting essentially of” refers to a composition in a formulation whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” can refer to compositions which contain the active ingredient and components which facilitate the release of the active ingredient. For example, the composition can contain one or more components that provide extended release of the active ingredient over time to the subject. In some embodiments, the term “consisting” refers to a composition, which contains the active ingredient and a pharmaceutically acceptable carrier or excipient.

As used herein, the term “nonsteroidal” and the phrase “nonsteroidal backbone” in the context of GRMs and SGRMs refers to GRMs and SGRMs that do not share structural homology to, or are not modifications of, cortisol with its steroid backbone containing seventeen carbon atoms, bonded in four fused rings. Such compounds include synthetic mimetics and analogs of proteins, including partially peptidic, pseudopeptidic and non-peptidic molecular entities.

Nonsteroidal GRA, SGRA, GRM, and SGRM compounds include compounds comprising a fused azadecalin structure (which may also be termed a fused azadecalin backbone), compounds comprising a heteroaryl ketone fused azadecalin structure (which may also be termed a heteroaryl ketone fused azadecalin backbone), compounds comprising an octahydro fused azadecalin structure (which may also be termed an octahydro fused azadecalin backbone). Exemplary nonsteroidal GRA, SGRA, GRM, and SGRM compounds comprising a fused azadecalin structure include those described in U.S. Pat. Nos. 7,928,237 and 8,461,172. Exemplary nonsteroidal GRA, SGRA, GRM, and SGRM compounds comprising a heteroaryl ketone fused azadecalin structure include those described in U.S. Pat. 8,859,774. Exemplary nonsteroidal GRA, SGRA, GRM, and SGRM compounds comprising an octahydro fused azadecalin structure include those described in U.S. Pat. 10,047,082. All patents, patent publications, and patent applications disclosed herein are hereby incorporated by reference in their entireties.

Where substituent groups are specified by their conventional chemical formulae, written from left to right, they equally encompass the chemically identical substituents that would result from writing the structure from right to left, e.g., —CH₂O— is equivalent to —OCH₂—.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, and hexyl.

“Alkoxy” refers to an alkyl group having an oxygen atom that connects the alkyl group to the point of attachment: alkyl-O-. As for the alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

“Halogen” refers to fluorine, chlorine, bromine, and iodine.

“Haloalkyl” refers to alkyl, as defined above, where some or all of the hydrogen atoms are replaced with halogen atoms. As for the alkyl group, haloalkyl groups can have any suitable number of carbon atoms, such as C₁₋₆, and include trifluoromethyl, fluoromethyl, etc.

The term “perfluoro” can be used to define a compound or radical where all the hydrogens are replaced with fluorine. For example, perfluoromethane includes 1,1,1-trifluoromethyl.

“Haloalkoxy” refers to an alkoxy group where some or all of the hydrogen atoms are substituted with halogen atoms. As for the alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, and perfluoroethoxy.

“Cycloalkyl” refers to a saturated or partially unsaturated, monocyclic, fused bicyclic, or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene, and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to, cyclopropyl, cyclobutyl, cyclopentyl, cyclohexyl, cycloheptyl, and cyclooctyl. When cycloalkyl is a saturated monocyclic C₃₋₆ cycloalkyl, exemplary groups include, but are not limited to, cyclopropyl, cyclobutyl, cyclopentyl, and cyclohexyl.

“Heterocycloalkyl” refers to a saturated ring system having from 3 to 12 ring members and from 1 to 4 heteroatoms of N, O, and S. Additional heteroatoms can also be useful, including but not limited to, B, Al, Si, and P. The heteroatoms can also be oxidized, such as, but not limited to, —S(O)— and —S(O)₂—. Heterocycloalkyl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3-and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxalidine, thiazolidine, isothiazolidine, dioxolane, dithiolane, morpholine, thiomorpholine, dioxane, or dithiane. The heterocycloalkyl groups can also be fused to aromatic or nonaromatic ring systems to form members including, but not limited to, indoline.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Aryl” refers to an aromatic ring system having any suitable number of ring atoms and any suitable number of rings. Aryl groups can include any suitable number of ring atoms, such as 6, 7, 8, 9, 10, 11, 12, 13, 14, 15, or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, that has a methylene linking group. Some aryl groups have from 6 to 12 ring members, such as phenyl, naphthyl, or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic, fused bicyclic, or tricyclic aromatic ring assembly containing 5 to 16 ring atoms, where from 1 to 5 of the ring atoms are a heteroatom such as N, O, or S. Additional heteroatoms can also be useful, including but not limited to, B, Al, Si, and P. The heteroatoms can also be oxidized, such as, but not limited to, N-oxide, —S(O)—, and —S(O)₂—. Heteroaryl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5; or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4-, and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2-, and 3-pyrrole; pyridine includes 2-, 3- and 4-pyridine; imidazole includes 1-, 2-, 4- and 5-imidazole; pyrazole includes 1-, 3-, 4- and 5-pyrazole; triazole includes 1-, 4- and 5-triazole; tetrazole includes 1- and 5-tetrazole; pyrimidine includes 2-, 4-, 5- and 6- pyrimidine; pyridazine includes 3- and 4-pyridazine; 1,2,3-triazine includes 4- and 5-triazine; 1,2,4-triazine includes 3-, 5- and 6-triazine; 1,3,5-triazine includes 2-triazine; thiophene includes 2- and 3-thiophene; furan includes 2- and 3-furan; thiazole includes 2-, 4-and 5-thiazole; isothiazole includes 3-, 4- and 5-isothiazole; oxazole includes 2-, 4- and 5-oxazole; isoxazole includes 3-, 4- and 5-isoxazole; indole includes 1-, 2- and 3-indole; isoindole includes 1- and 2-isoindole; quinoline includes 2-, 3- and 4-quinoline; isoquinoline includes 1-, 3- and 4-isoquinoline; quinazoline includes 2- and 4-quinoazoline; cinnoline includes 3- and 4-cinnoline; benzothiophene includes 2- and 3-benzothiophene; and benzofuran includes 2- and 3-benzofuran.

Some heteroaryl groups include those having from 5 to 10 ring members and from 1 to 3 ring atoms including N, O, or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring heteroatoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Heteroatoms” refers to O, S, or N.

“Salt” refers to acid or base salts of the compounds used in the methods of the present invention. Illustrative examples of pharmaceutically-acceptable salts are mineral acid (hydrochloric acid, hydrobromic acid, phosphoric acid, and the like) salts, organic acid (acetic acid, propionic acid, glutamic acid, citric acid, and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington’s Pharmaceutical Sciences, 17th ed., Mack Publishing Company, Easton, Pa., 1985, which is incorporated herein by reference.

“Isomers” refers to compounds with the same chemical formula but which are structurally distinguishable.

“Tautomer” refers to one of two or more structural isomers which exist in equilibrium and which are readily converted from one form to another.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to produce compounds which are not inherently unstable - and/or would be known to one of ordinary skill in the art as likely to be unstable under ambient conditions - such as aqueous, neutral, or physiological conditions.

“Pharmaceutically acceptable excipient” and “pharmaceutically acceptable carrier” refer to a substance that aids the administration of an active agent to - and absorption by - a subject and can be included in the compositions of the present invention without causing a significant adverse toxicological effect on the patient. As used herein, these terms are intended to include any and all solvents, dispersion media, coatings, antibacterial and antifungal agents, antioxidant agents, isotonic and absorption delaying agents, and the like, compatible with pharmaceutical administration. Non-limiting examples of pharmaceutically acceptable excipients include water, NaCl, normal saline solutions, lactated Ringer’s, normal sucrose, normal glucose, binders, fillers, disintegrants, encapsulating agents, plasticizers, lubricants, coatings, sweeteners, flavors and colors, and the like. One of ordinary skill in the art will recognize that other pharmaceutical excipients are useful in the present invention. The use of such media and agents for pharmaceutically active substances is well known in the art. Except insofar as any conventional media or agent is incompatible with the active compound, use thereof in the compositions is contemplated. Supplementary active compounds can also be incorporated into the compositions. One of ordinary skill in the art will recognize that other pharmaceutical excipients are useful in the present invention.

In some embodiments, the methods disclosed herein include combination therapies which include administering a GRM comprising a fused azadecalin structure; a GRM comprising a heteroaryl ketone fused azadecalin structure; or a GRM comprising an octahydro fused azadecalin structure.

Exemplary GRMs comprising a fused azadecalin structure include those described in U.S. Pat. Nos. 7,928,237; and 8,461,172 and can be prepared as disclosed therein. These patents are incorporated herein in their entirety. Such exemplary GRMs may be SGRMs. In some cases, the GRM comprising a fused azadecalin structure has the following structure:

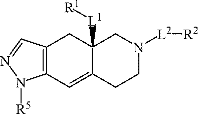

wherein
L¹ and L² are members independently selected from a bond and unsubstituted alkylene;
R¹ is a member selected from unsubstituted alkyl, unsubstituted heteroalkyl, unsubstituted heterocycloalkyl, -OR^{1A}, -NR^{1C}R^{1D}, -C(O)NR^{1C}R^{1D}, and -C(O)OR^{1A}, wherein
R^{1A} is a member selected from hydrogen, unsubstituted alkyl and unsubstituted heteroalkyl,
R^{1C} and R^{1D} are members independently selected from unsubstituted alkyl and unsubstituted heteroalkyl,
wherein R^{1C} and R^{1D} are optionally joined to form an unsubstituted ring with the nitrogen to which they are attached, wherein said ring optionally comprises an additional ring nitrogen;
R² has the formula:

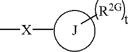

wherein
   R^{2G} is a member selected from hydrogen, halogen, unsubstituted alkyl, unsubstituted heteroalkyl, unsubstituted cycloalkyl, unsubstituted heterocycloalkyl, —CN, and —CF₃;
   J is phenyl;
   t is an integer from 0 to 5;
   X is —S(O₂)—; and
   R⁵ is phenyl optionally substituted with 1-5 R^{5A} groups, wherein
   R^{5A} is a member selected from hydrogen, halogen, -OR^{5A1}, -S(O₂)NR^{5A2}R^{5A3}, —CN, and unsubstituted alkyl, wherein
   R^{5A1} is a member selected from hydrogen and unsubstituted alkyl, and
   R^{5A2} and R^{5A3} are members independently selected from hydrogen and unsubstituted alkyl,
   or salts and isomers thereof.

In some cases, the fused azadecalin compound is

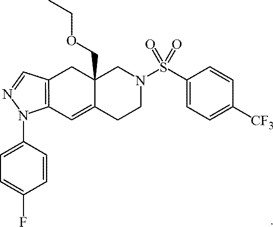

Exemplary GRMs comprising a heteroaryl ketone fused azadecalin structure include those described in U.S. 8,859,774, which can be prepared as disclosed therein, and is incorporated herein in its entirety. Such exemplary GRMs may be SGRMs. In some cases, the GRM comprising a heteroaryl ketone fused azadecalin structure has the following structure:

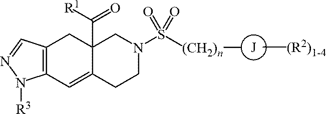

wherein
R¹ is a heteroaryl ring having from 5 to 6 ring members and from 1 to 4 heteroatoms each independently selected from the group consisting of N, O and S, optionally substituted with 1-4 groups each independently selected from R^{1ª};
each R^{1a} is independently selected from the group consisting of hydrogen, C₁₋₆ alkyl, halogen, C₁₋₆ haloalkyl, C₁₋₆ alkoxy, C₁₋₆ haloalkoxy, —CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, -NR^{2a}R^{2b}, -C(O)R^{2a}, -C(O)OR^{2a}, -C(O)NR^{2a}R^{2b}, -SR^{2a}, -S(O)R^{2a}, -S( O)₂R^{2a}, C₃₋₈ cycloalkyl, and C₃₋₈ heterocycloalkyl, wherein the heterocycloalkyl groups are optionally substituted with 1-4 R^{2c} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, —CN, and -NR^{2a}R^{2b},
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl; and
subscript n is an integer from 0 to 3;
or salts and isomers thereof.

In some cases, the nonsteroidal SGRM is CORT125134, i.e., (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, which has the following structure:

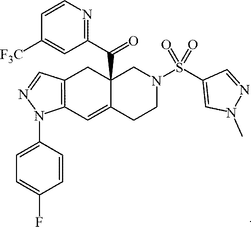

Exemplary GRMs comprising an octahydro fused azadecalin structure include those described in U.S. 10,047,082 and can be prepared as described therein, the disclosure of which U.S. Pat. is incorporated herein in its entirety. Such exemplary GRMs may be SGRMs. In some cases, the GRM comprising an octahydro fused azadecalin structure has the following structure:

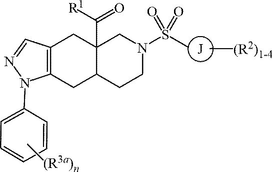

wherein
R¹ is a heteroaryl ring having from 5 to 6 ring members and from 1 to 4 heteroatoms each independently selected from the group consisting of N, O and S, optionally substituted with 1-4 groups each independently selected from R^{1a},
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl;
ring J is selected from the group consisting of an aryl ring and a heteroaryl ring having from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, -NR^{2a}R^{2b}, -C(O)R^{2a}, -C(O)OR^{2a}, -C(O)NR^{2a}R^{2b}, -SR^{2a}, -S(O)R^{2a}, -S( O)₂R^{2a}, C₃₋₈ cycloalkyl, and C₃₋₈ heterocycloalkyl having from 1 to 3 heteroatoms each independently selected from the group consisting of N, O and S;
alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups;
R^{2a}, R^{2b} and R^{2c} are each independently selected from the group consisting of hydrogen and C₁₋₆ alkyl;
each R^{3a} is independently halogen; and
subscript n is an integer from 0 to 3;
or salts and isomers thereof.

In embodiments, the octahydro fused azadecalin compound has the formula:

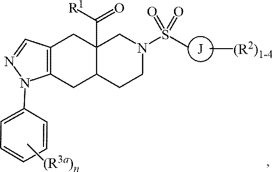

wherein R¹ is selected from the group consisting of pyridine and thiazole, optionally substituted with 1-4 groups each independently selected from R^{1a}; each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl; ring J is selected from the group consisting of phenyl, pyridine, pyrazole, and triazole; each R² is independently selected from the group consisting of hydrogen, C₁₋₆ alkyl, halogen, C₁₋₆ haloalkyl, and —CN; R^{3a} is F; subscript n is an integer from 0 to 3; or salts and isomers thereof.

In some cases, the nonsteroidal SGRM is exicorilant (also termed CORT125281), i.e., ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone, which has the following structure:

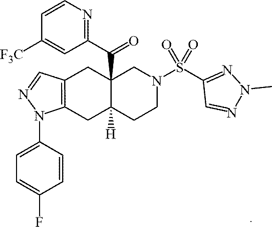

In some cases, the nonsteroidal SGRM is CORT125329, i.e., ((4aR,8aS)-1-(4-fluorophenyl)-6-((2-isopropyl-2H-1,2,3-triazol-4-yl)sulfonyl)-4,4a,5,6,7,8,8a,9-octahydro-1H-pyrazolo[3,4-g]isoquinolin-4a-yl)(thiazol-2-yl)methanone, which has the following structure:

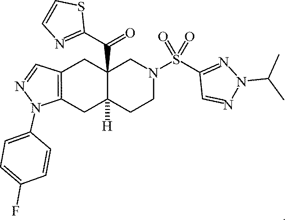

### Identifying Selective Glucocorticoid Receptor Modulators (SGRMS)

To determine whether a test compound is a SGRM, the compound is first subjected to assays to measure its ability to bind to the GR and inhibit GR-mediated activities, which determines whether the compound is a glucocorticoid receptor modulator. The compound, if confirmed to be a glucocorticoid receptor modulator, is then subjected to a selectivity test to determine whether the compound can bind specifically to GR as compared to non GR proteins, such as the estrogen receptor, the progesterone receptor, the androgen receptor, or the mineralocorticoid receptor. In one embodiment, a SGRM binds to GR at a substantially higher affinity, e.g., at least 10 times higher affinity, than to non-GR proteins. A SGRM may exhibit a 100-fold, 1000-fold or greater selectivity for binding to GR relative to binding to non-GR proteins.

### Binding

A test compounds’ ability to bind to the glucocorticoid receptor can be measured using a variety of assays, for example, by screening for the ability of the test compound to compete with a glucocorticoid receptor ligand, such as dexamethasone, for binding to the glucocorticoid receptor. Those of skill in the art will recognize that there are a number of ways to perform such competitive binding assays. In some embodiments, the glucocorticoid receptor is pre-incubated with a labeled glucocorticoid receptor ligand and then contacted with a test compound. This type of competitive binding assay may also be referred to herein as a binding displacement assay. A decrease of the quantity of labeled ligand bound to glucocorticoid receptor indicates that the test compound binds to the glucocorticoid receptor. In some cases, the labeled ligand is a fluorescently labeled compound (e.g., a fluorescently labeled steroid or steroid analog). Alternatively, the binding of a test compound to the glucocorticoid receptor can be measured directly with a labeled test compound. This latter type of assay is called a direct binding assay.

Both direct binding assays and competitive binding assays can be used in a variety of different formats. The formats may be similar to those used in immunoassays and receptor binding assays. For a description of different formats for binding assays, including competitive binding assays and direct binding assays, see Basic and Clinical Immunology 7th Edition (D. Stites and A. Terr ed.) 1991; Enzyme Immunoassay, E.T. Maggio, ed., CRC Press, Boca Raton, Florida (1980); and “Practice and Theory of Enzyme Immunoassays,” P. Tijssen, Laboratory Techniques in Biochemistry and Molecular Biology, Elsevier Science Publishers B.V. Amsterdam (1985), each of which is incorporated herein by reference.

In solid phase competitive binding assays, for example, the sample compound can compete with a labeled analyte for specific binding sites on a binding agent bound to a solid surface. In this type of format, the labeled analyte can be a glucocorticoid receptor ligand and the binding agent can be glucocorticoid receptor bound to a solid phase. Alternatively, the labeled analyte can be labeled glucocorticoid receptor and the binding agent can be a solid phase glucocorticoid receptor ligand. The concentration of labeled analyte bound to the capture agent is inversely proportional to the ability of a test compound to compete in the binding assay.

Alternatively, the competitive binding assay may be conducted in the liquid phase, and any of a variety of techniques known in the art may be used to separate the bound labeled protein from the unbound labeled protein. For example, several procedures have been developed for distinguishing between bound ligand and excess bound ligand or between bound test compound and the excess unbound test compound. These include identification of the bound complex by sedimentation in sucrose gradients, gel electrophoresis, or gel isoelectric focusing; precipitation of the receptor-ligand complex with protamine sulfate or adsorption on hydroxylapatite; and the removal of unbound compounds or ligands by adsorption on dextran-coated charcoal (DCC) or binding to immobilized antibody. Following separation, the amount of bound ligand or test compound is determined.

Alternatively, a homogenous binding assay may be performed in which a separation step is not needed. For example, a label on the glucocorticoid receptor may be altered by the binding of the glucocorticoid receptor to its ligand or test compound. This alteration in the labeled glucocorticoid receptor results in a decrease or increase in the signal emitted by label, so that measurement of the label at the end of the binding assay allows for detection or quantitation of the glucocorticoid receptor in the bound state. A wide variety of labels may be used. The component may be labeled by any one of several methods. Useful radioactive labels include those incorporating ³H, ¹²⁵I, ³⁵S, ¹⁴C, or ³²P. Useful non-radioactive labels include those incorporating fluorophores, chemiluminescent agents, phosphorescent agents, electrochemiluminescent agents, and the like. Fluorescent agents are especially useful in analytical techniques that are used to detect shifts in protein structure such as fluorescence anisotropy and/or fluorescence polarization. The choice of label depends on sensitivity required, ease of conjugation with the compound, stability requirements, and available instrumentation. For a review of various labeling or signal producing systems which may be used, see U.S. Pat. No. 4,391,904, which is incorporated herein by reference in its entirety for all purposes. The label may be coupled directly or indirectly to the desired component of the assay according to methods well known in the art. In some cases, a test compound is contacted with a GR in the presence of a fluorescently labeled ligand (e.g., a steroid or steroid analog) with a known affinity for the GR, and the quantity of bound and free labeled ligand is estimated by measuring the fluorescence polarization of the labeled ligand.

### Activity

### 1) HepG2 Tyrosine Aminotransferase (TAT) Assay

Compounds that have demonstrated the desired binding affinity to GR are tested for their activity in inhibiting GR mediated activities. The compounds are typically subject to a Tyrosine Aminotransferase Assay (TAT assay), which assesses the ability of a test compound to inhibit the induction of tyrosine aminotransferase activity by dexamethasone. See Example 1. GR modulators that are suitable for the method disclosed herein have an IC₅₀ (half maximal inhibition concentration) of less than 10 micromolar. Other assays, including but not limited to those described below, can also be deployed to confirm the GR modulation activity of the compounds.

### 2) Cell-Based Assays

Cell-based assays which involve whole cells or cell fractions containing glucocorticoid receptors can also be used to assay for a test compound’s binding or modulation of activity of the glucocorticoid receptor. Exemplary cell types that can be used according to the methods of the invention include, e.g., any mammalian cells including leukocytes such as neutrophils, monocytes, macrophages, eosinophils, basophils, mast cells, and lymphocytes, such as T cells and B cells, leukemia cells, Burkitt’s lymphoma cells, tumor cells (including mouse mammary tumor virus cells), endothelial cells, fibroblasts, cardiac cells, muscle cells, breast tumor cells, ovarian cancer carcinomas, cervical carcinomas, glioblastomas, liver cells, kidney cells, and neuronal cells, as well as fungal cells, including yeast. Cells can be primary cells or tumor cells or other types of immortal cell lines. Of course, the glucocorticoid receptor can be expressed in cells that do not express an endogenous version of the glucocorticoid receptor.

In some cases, fragments of the glucocorticoid receptor, as well as protein fusions, can be used for screening. When molecules that compete for binding with the glucocorticoid receptor ligands are desired, the GR fragments used are fragments capable of binding the ligands (e.g., dexamethasone). Alternatively, any fragment of GR can be used as a target to identify molecules that bind the glucocorticoid receptor. Glucocorticoid receptor fragments can include any fragment of, e.g., at least 20, 30, 40, 50 amino acids up to a protein containing all but one amino acid of glucocorticoid receptor.

In some embodiments, a reduction in signaling triggered by glucocorticoid receptor activation is used to identify glucocorticoid receptor modulators. Signaling activity of the glucocorticoid receptor can be determined in many ways. For example, downstream molecular events can be monitored to determine signaling activity. Downstream events include those activities or manifestations that occur as a result of stimulation of a glucocorticoid receptor. Exemplary downstream events useful in the functional evaluation of transcriptional activation and antagonism in unaltered cells include upregulation of a number of glucocorticoid response element (GRE)-dependent genes (PEPCK, tyrosine amino transferase, aromatase). In addition, specific cell types susceptible to GR activation may be used, such as osteocalcin expression in osteoblasts which is downregulated by glucocorticoids; primary hepatocytes which exhibit glucocorticoid mediated upregulation of PEPCK and glucose-6-phosphate (G-6-Pase)). GRE-mediated gene expression has also been demonstrated in transfected cell lines using well-known GRE-regulated sequences (e.g., the mouse mammary tumor virus promoter (MMTV) transfected upstream of a reporter gene construct). Examples of useful reporter gene constructs include luciferase (luc), alkaline phosphatase (ALP) and chloramphenicol acetyl transferase (CAT). The functional evaluation of transcriptional repression can be carried out in cell lines such as monocytes or human skin fibroblasts. Useful functional assays include those that measure IL-1beta stimulated IL-6 expression; the downregulation of collagenase, cyclooxygenase-2 and various chemokines (MCP-1, RANTES); LPS stimulated cytokine release, e.g., TNFα; or expression of genes regulated by NFkB or AP-1 transcription factors in transfected cell-lines.

Compounds that are tested in whole-cell assays can also be tested in a cytotoxicity assay. Cytotoxicity assays are used to determine the extent to which a perceived effect is due to non- glucocorticoid receptor binding cellular effects. In an exemplary embodiment, the cytotoxicity assay includes contacting a constitutively active cell with the test compound. Any decrease in cellular activity indicates a cytotoxic effect.

### 3) Additional Assays

Further illustrative of the many assays which can be used to identify compositions utilized in the methods of the invention, are assays based on glucocorticoid activities in vivo. For example, assays that assess the ability of a putative GR modulator to inhibit uptake of 3H-thymidine into DNA in cells which are stimulated by glucocorticoids can be used. Alternatively, the putative GR modulator can complete with 3H-dexamethasone for binding to a hepatoma tissue culture GR (see, e.g., Choi, et al., Steroids 57:313-318, 1992). As another example, the ability of a putative GR modulator to block nuclear binding of 3H-dexamethasone-GR complex can be used (Alexandrova et al., J. Steroid Biochem. Mol. Biol. 41:723-725, 1992). To further identify putative GR modulators, kinetic assays able to discriminate between glucocorticoid agonists and modulators by means of receptor-binding kinetics can also be used (as described in Jones, Biochem J. 204:721-729, 1982).

In another illustrative example, the assay described by Daune, Molec. Pharm. 13:948-955, 1977; and in U.S. Pat. No. 4,386,085, can be used to identify anti-glucocorticoid activity. Briefly, the thymocytes of adrenalectomized rats are incubated in nutritive medium containing dexamethasone with the test compound (the putative GR modulator) at varying concentrations. ³H-uridine is added to the cell culture, which is further incubated, and the extent of incorporation of radiolabel into polynucleotide is measured. Glucocorticoid agonists decrease the amount of ³H-uridine incorporated. Thus, a GR modulator will oppose this effect.

### Selectivity

The GR modulators selected above are then subject to a selectivity assay to determine whether they are SGRMs. Typically, selectivity assays include testing a compound that binds glucocorticoid receptor in vitro for the degree of binding to non-glucocorticoid receptor proteins. Selectivity assays may be performed in vitro or in cell-based systems, as described above. Binding may be tested against any appropriate non-glucocorticoid receptor protein, including antibodies, receptors, enzymes, and the like. In an exemplary embodiment, the non- glucocorticoid receptor binding protein is a cell-surface receptor or nuclear receptor. In another exemplary embodiment, the non- glucocorticoid receptor protein is a steroid receptor, such as estrogen receptor, progesterone receptor, androgen receptor, or mineralocorticoid receptor.

The selectivity of the antagonist for the GR relative to the MR can be measured using a variety of assays known to those of skill in the art. For example, specific antagonists can be identified by measuring the ability of the antagonist to bind to the GR compared to the MR (see, e.g., U.S. Pat. Nos. 5,606,021; 5,696,127; 5,215,916; 5,071,773). Such an analysis can be performed using either a direct binding assay or by assessing competitive binding to the purified GR or MR in the presence of a known ligand. In an exemplary assay, cells that stably express the glucocorticoid receptor or mineralocorticoid receptor (see, e.g., U.S. Pat. No. 5,606,021) at high levels are used as a source of purified receptor. The affinity of the ligand for the receptor is then directly measured. Those GR modulators that exhibit at least a 10-fold, 100-fold higher affinity, often 1000-fold, for the GR relative to the MR are then selected for use in the methods of the invention.

The selectivity assay may also include assaying the ability to inhibit GR-mediated activities, but not MR-mediated activities. One method of identifying such a GR-specific modulator is to assess the ability of an antagonist to prevent activation of reporter constructs using transfection assays (see, e.g., Bocquel et al, J. Steroid Biochem Molec. Biol. 45:205-215, 1993; U.S. Pat. Nos. 5,606,021, 5,929,058). In an exemplary transfection assay, an expression plasmid encoding the receptor and a reporter plasmid containing a reporter gene linked to receptor-specific regulatory elements are cotransfected into suitable receptor-negative host cells. The transfected host cells are then cultured in the presence and absence of a hormone, such as cortisol or an analog thereof, able to activate the hormone responsive promoter/enhancer element of the reporter plasmid. Next the transfected and cultured host cells are monitored for induction (i.e., the presence) of the product of the reporter gene sequence. Finally, the expression and/or steroid binding-capacity of the hormone receptor protein (coded for by the receptor DNA sequence on the expression plasmid and produced in the transfected and cultured host cells), is measured by determining the activity of the reporter gene in the presence and absence of an antagonist. The antagonist activity of a compound may be determined in comparison to known antagonists of the GR and MR receptors (see, e.g., U.S. Pat. No. 5,696,127). Efficacy is then reported as the percent maximal response observed for each compound relative to a reference antagonist compound. GR modulators that exhibits at least a 100-fold, often 1000-fold or greater, activity towards the GR relative to the MR, PR, or AR are then selected for use in the methods disclosed herein.

### Diagnosing Cancer

Cancers are characterized by uncontrolled growth and/or spread of abnormal cells. A biopsy is tyically taken and the cell or tissue from the biopsy is examined under a microscope in order to confirm a suspected condition. In some cases, additional tests need to be performed on the cells’ proteins, DNA, and RNA to verify the diagnosis.

### Identifying Checkpoint Inhibitor Sensitive Cancer

In some embodiments of the invention, methods are used to treat patients having at least one checkpoint inhibitor sensitive cancer. Checkpoint inhibitor sensitive cancers are those that are responsive to checkpoint inhibitors, i.e., administration of one or more checkpoint inhibitors can reduce tumor load or achieve beneficial or desired clinical results related to cancer improvement. For example, the administration of the checkpoint inhibitor may bring about one or more of the following: reducing the number of cancer cells; reducing the tumor size; inhibiting (i.e., slowing to some extent and/or stop) cancer cell infiltration into peripheral organs; inhibiting (i.e., slowing to some extent and/or stop) tumor metastasis; inhibiting, to some extent, tumor growth; and/or relieving to some extent one or more of the symptoms associated with the disorder; shrinking the size of the tumor; decreasing symptoms resulting from the disease; increasing the quality of life of those suffering from the disease; decreasing the dose of other medications required to treat the disease; delaying the progression of the disease; and/or prolonging survival of patients.

Checkpoint inhibitor sensitive tumors often have high expression of ligands, e.g., PD-L1 or B7, that bind to checkpoint proteins, PD-1 or CTLA-4, respectively. These interactions suppress immune responses against the tumor cells. It is believed that administration of a GRM or SGRM, as disclosed herein, may induce checkpoint-inhibitor sensitivity in a tumor otherwise relatively insensitive to checkpoint inhibitors, or may enhance checkpoint-inhibitor sensitivity in a tumor. Non-limiting examples of checkpoint-inhibitor-sensitive tumors, and tumors which may be induced to become checkpoint-inhibitor sensitive, include lung cancer, liver cancer, ovarian cancer, cervical cancer, skin cancer, bladder cancer, colon cancer, breast cancer, glioma, renal carcinoma, stomach cancer, esophageal cancer, oral squamous cell cancer, head/neck cancer, melanoma, sarcoma, renal cell tumor, hepatocellular tumor, glioblastoma, neuroendocrine tumor, bladder cancer, pancreatic cancer, gall bladder cancer, gastric cancer, prostate cancer, endometrial cancer, thyroid cancer and mesothelioma.

### III. Identifying GR Expression

In some embodiments, the checkpoint inhibitor sensitive cancer is also a GR⁺ cancer. GR expression in cancer cells can be examined by using one or more of the routine biochemical analyses. In some embodiments, GR expression is determined by detecting GR transcript expression, using methods such as microarray and RT-PCR. In other embodiments, GR expression is determined by detecting protein expression, using methods such as, western blot analysis and immunohistochemistry staining. In yet other embodiments, the GR expression is determined using a combination of these methods.

In a preferred embodiment, immunohistochemistry staining is performed and a H-score method is used to quantify the expression of GR on cancer tissues. In one exemplar assay, Formalin-fixed, paraffin-embedded tumor tissue sections are deparaffinized and treated with antigen retrieval solution to render the glucocorticoid receptors readily accessible to anti-GR antibodies. Anti-GR antibodies are then incubated with the tissue sections and the antibodies bound to the GR on the tissue sections are detected by addition of a horse peroxidase (HRP) conjugated secondary antibody that recognizes the anti-GR antibody. The HRP on the secondary antibody conjugate catalyzes a colorimetric reaction and upon contacting the appropriate substrate, produces a staining in the locations where GR is present. In one approach, the intensity level of the GR staining is represented by 0 for negative staining, 1+ for weak staining, 2+ for moderate staining, and 3+ for strong staining. See www.ihcworld.com/ihc_scoring.htm. The percentage of GR⁺ cells of each intensity level is multiplied with the intensity level, and the results for all intensity levels are summed to generate a H-score between 0-300. In one embodiment, the cancer type having a H-score equal to or higher than a predetermined threshold is considered GR⁺ cancer. In a preferred embodiment, the threshold is 150. In another embodiment, a GR⁺ cancer is one that has at least 10% tumor cells showing GR staining at any intensity. A number of cancer types are GR⁺, using the threshold of H-score 150. See Table 1, below. A majority of these cancer types are also checkpoint inhibitor sensitive cancers as shown by published results of clinical trials. See, the web-site “clinicaltrials.gov”.

### Checkpoint Inhibitors

The method disclosed herein uses at least one SGRM in combination with at least one checkpoint inhibitor to treat cancers. In some embodiments, the checkpoint inhibitor is an antibody (“CIA”) against at least one checkpoint protein. In some embodiments, the checkpoint inhibitor is a small molecule, non-protein compound (“CIC”) that blocks the immunosuppression pathway induced by one or more checkpoint proteins.

### I. Checkpoint Inhibitor Antibodies (“CIA”)

In one embodiment, the method for treating cancer comprises administering a SGRM in combination with a checkpoint inhibitor antibody. Such an antibody can block the immunosuppression activity of the checkpoint protein. A number of such antibodies have already been shown to be effective in treating cancers, e.g., antibodies against PD-1, CTLA4, and PD-L1.

Anti-PD-1 antibodies have been used for the treatment of melanoma, non-small-cell lung cancer, bladder cancer, prostate cancer, colorectal cancer, head and neck cancer, triple-negative breast cancer, leukemia, lymphoma and renal cell cancer. Exemplary anti-PD-1 antibodies include lambrolizumab (MK-3475, MERCK), nivolumab (BMS-936558, BRISTOL-MYERS SQUIBB), AMP-224 (MERCK), and pidilizumab (CT-011, CURETECH LTD.).

Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies. Exemplary anti-PD-L1 antibodies include MDX-1105 (MEDAREX), MEDI4736 (MEDIMMUNE), MPDL3280A (GENENTECH) and BMS-936559 (BRISTOL-MYERS SQUIBB).

Anti-CTLA4 antibodies have been used in clinical trials for the treatment of melanoma, prostate cancer, small cell lung cancer, non-small cell lung cancer. A significant feature of anti-CTL4A is the kinetics of anti-tumor effect, with a lag period of up to 6 months after initial treatment required for physiologic response. In some cases, tumors may actually increase in size after treatment initiation, before a reduction is seen (Pardoll, 2012, Nature Reviews Cancer 12:252-264). Exemplary anti-CTLA4 CIAs include ipilimumab (Bristol-Myers Squibb) and tremelimumab (PFIZER).

CIAs against other checkpoint proteins, such as LAG3, B7-H3, B7-H4 and TIM3, may also be used in combination with the SGRMs disclosed herein to treat cancers.

The CIAs used in this disclosure can be a combination of different CIAs, especially if the target checkpoint proteins, e.g., PD-1 and CTLA4, suppress immune response via different signaling pathways. Thus a combination of CIAs against either of the checkpoint proteins or a single CIA that is against both checkpoint proteins may provide an enhanced immune response.

### Generating CIAs

CIAs can be developed using methods well known in the art. See, for example, Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991). Monoclonal antibodies can be obtained by injecting mice with a composition comprising an antigen, e.g. a checkpoint protein or an epitope of thereof, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

Monoclonal antibodies produced can be isolated and purified from hybridoma cultures by a variety of well-established techniques. Such isolation techniques include affinity chromatography with Protein-A Sepharose, size-exclusion chromatography, and ionexchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992). After the initial raising of antibodies to a checkpoint protein, the antibodies can be sequenced and subsequently prepared by recombinant techniques. Humanization and chimerization of murine antibodies and antibody fragments are well known to those skilled in the art. See, for example, Leung et al. Hybridoma 13:469 (1994); US20140099254 A1.

Human antibodies can be produced using transgenic mice that have been genetically engineered to produce specific human antibodies in response to antigenic challenge using a checkpoint protein. See Green et al., Nature Genet. 7: 13 (1994), Lonberg et al., Nature 368:856 (1994). Human antibodies against a checkpoint protein also can be constructed by genetic or chromosomal trandfection methods, phage display technology, or by in vitro activated B cells. See e.g., McCafferty et al., 1990, Nature 348: 552-553; U.S. Pat. Nos. 5, 567,610 and 5, 229,275.

### Modifying CIAs

CIAs may also be produced by introducing conservative modifications relative to the existing CIAs. For example, a modifed CIA may comprise heavy and light chain variable regions, and/or a Fc region that are homologous to the counterparts of an antibody produced above. The modified CIA that can be used for the method disclosed herein must retain the desired functional properties of being able to block the checkpoint signaling pathway.

CIAs may also be produced by altering protein modification sites. For example, sites of glycosylation of the antibody can be altered to produce an antibody lacking glycosylation and the so modified CIAs typically have increased affinity of the antibody for antigen. Antibodies can also be pegylated by reacting with polyethylene glycol (PEG) under conditions in which one or more PEG groups become attached to the antibody. Pegylation can increase the biological half-life of the antibody. Antibodies having such modifications can also be used in combination with the selective GR modulator disclosed herein so long as it retains the desired functional properties of blocking the checkpoint pathways.

### II. Small Molecule, Non-Protein Checkpoint Inhibitor Compounds (“CICs”)

In another embodiment, the method for treating cancer, e. g. a checkpoint inhibitor sensitive cancer, uses a SGRM in combination with a CIC. A CIC is a small molecule, non-protein compound that antagonizes a checkpoint protein’s immune suppression function. Many CICs are known in the art, for example, those disclosed in PCT Publications WO2015034820, WO20130144704, and WO2011082400.

CICs can also be identified using any of the numerous approaches in combinatorial library methods known in the art and disclosed in, e.g., European Patent Application EP2360254. The cominatorial libraries include: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

### III. Evaluating the Functional Properties of the Candidate Checkpoint Inhibitors

A number of well-known assays can be used to assess whether a candidate, i.e., an antibody generated by immunizing an animal with an antigen comprising a checkpoint protein, an epitope of the checkpoint protein, or a test compound from combinatorial libraries, as disclosed above, is a checkpoint inhibitor. Non-limiting exemplar assays include binding assays -- such as Enzyme-Linked Immunosorbent Assays (ELISAs), radioimmunoassays (RIA) --, Fluorescence-Activated Cell Sorting (FACS) analysis, cell-based assays, and in vivo assays.

### Binding Assays

In one embodiment, the assay is a direct binding assay. The checkpoint protein can be coupled with a radioisotope or enzymatic label such that binding of the checkpoint protein and the candidate can be determined by detecting the labeled checkpoint protein in a complex. For example, a checkpoint protein can be labeled with ¹²⁵I, ³⁵S, ¹⁴C, or ³H, either directly or indirectly, and the radioisotope detected by direct counting of radio-emission or by scintillation counting. Determining the ability of candidates to bind their cognate checkpoint protein can be accomplished, e.g., by measuring direct binding. Alternatively, checkpoint protein molecules can be enzymatically labeled with, for example, horseradish peroxidase, alkaline phosphatase, or luciferase, and binding of the candidates to the target checkpoint protein is determined by conversion of an appropriate substrate to product.

Enzyme-linked immunosorbent assay (ELISA) are commonly used to evaluate a CIA candidate’s binding specificity to its target checkpoint protein. In a typical assay, microtiter plates are coated with the checkpoint protein by coating overnight at 37° C. with 5 µg/ml checkpoint protein. Serum samples comprising candidate CIAs are diluted in PBS, 5% serum, 0.5% Tween-20 and are incubated in wells for 1 hour at room temperature, followed by the addition of anti-human IgG Fc and IgG F(ab′)-horseradish peroxidase in the same diluent. After 1 hour at room temperature enzyme activity is assessed by addition of ABTS substrate (Sigma, St. Louis Mo.) and read after 30 minutes at 415-490 nm.

The binding kinetics (e.g., binding affinity) of the candidates also can be assessed by standard assays known in the art, such as by Biacore analysis (Biacore AB, Uppsala, Sweden). In one exemplary assay, a purified recombinant human checkpoint protein is covalently linked to a CM5 chip (carboxy methyl dextran coated chip) via primary amines, using standard amine coupling chemistry and kit provided by Biacore. Binding is measured by flowing the candidates in HBS EP buffer (provided by Biacore AB) at a concentration of 267 nM at a flow rate of 50 µl/min. The checkpoint protein- candidate association kinetics are followed for 3 minutes and the dissociation kinetics are followed for 7 minutes. The association and dissociation curves are fitted to a 1:1 Langmuir binding model using BIA evaluation software (Biacore AB). To minimize the effects of avidity in the estimation of the binding constants, only the initial segment of data corresponding to association and dissociation phases are used for fitting. The K_{D}, Kₒₙ and K_{off} values of the interaction can be measured. Preferred checkpoint inhibitors can bind to their target checkpoint protein with a Kd of 1 ×10⁻⁷ M or less

For checkpoint proteins that block immune responses through binding to a ligand, additional binding assays may be employed to test for the ability of the candidate to block binding of the ligands to the checkpoint protein. In one exemplary assay, flow cytometry is used to test the blocking of the binding of the ligand (e.g., PD-L1) to the checkpoint protein (e.g., PD-1) expressed on transfected CHO cells. Various concentrations of the candidate are added to the suspension of cells expressing the checkpoint protein and incubated at 4° C. for 30 minutes. Unbound inhibitor is washed off and FITC-labeled ligand protein is added into the tubes and incubated at 4° C. for 30 minutes. FACS analysis is performed using a FACScan flow cytometer (Becton Dickinson, San Jose, Calif.). The mean fluorescent intensity (MFI) of staining of the cells indicates the amount of ligand that is bound to the checkpoint proteins. A reduced MFI in the sample to which the candidate is added indicates that the candidate is effective in blocking the binding of the ligand to the target checkpoint protein.

Homogenous Time-Resolved Fluorescence (HTRF) binding assay, such as described in PCT Publication WO2015034820, can also be used to assay the candidate’s ability to block the checkpoint protein-ligand interaction. In one embodiment, the CICs used in the method can inhibit the PD-1/PD-L1 interaction with IC₅₀ values of 10 pM or less, for example, from 0.01 to 10 pM, preferrably, 1 pM or less, e.g., from 0.01 to 1 pM, as measured by the PD-1/PD-L1 Homogenous Time-Resolved Fluorescence (HTRF) binding assay.

### Cell Based Assays

In another embodiment, the assay to evaluate whether a candidate is a checkpoint inhibitor is a cell-based assay. The Mixed Lymphocyte Reaction (MLR) assay, as described in U.S. Pat. No. 8,008,449, is routinely used to measure T cell proliferation, production of IL-2 and/or IFN-Y. In one exemplary assay, human T cells are purified from PBMCs using a human CD4⁺ T cell enrichment column (R&D systems). A candidate is added to a number of T cell cultures at different concentrations. The cells are cultured for 5 days at 37° C. and 100 µl of medium is taken from each culture for cytokine measurement. The levels of IFN-gamma and other cytokines are measured using OptEIA ELISA kits (BD Biosciences). The cells are labeled with ³H-thymidine, cultured for another 18 hours, and analyzed for cell proliferation. Results showing that, as compared to control, the culture containing the candidate shows increased T cell proliferation, increased production of IL-2, and/or IFN-gamma indicate the candidate is effective in blocking checkpoint protein’s inhibition of T cell immune response.

### In Vivo Assays

In another embodiment, the assay used to evaluate whether a candidate is a checkpoint inhibitor is an in vivo assay. In one exemplary assay, female AJ mice between 6-8 weeks of age (Harlan Laboratories) are randomized by weight into 6 groups. The mice are implanted subcutaneously in the right flank with 2×10⁶ SA1/N fibrosarcoma cells dissolved in 200 µl of DMEM media on day 0. The mice are treated with PBS vehicle, or the candidate at a predetermined dosage. The animals are dosed by intraperitoneal injection with approximately 200 µl of PBS containing the candidate or vehicle on days 1, 4, 8 and 11. The mice are monitored twice weekly for tumor growth for approximately 6 weeks. Using an electronic caliper, the tumors are measured three dimensionally (height×width×length) and tumor volume is calculated. Mice are euthanized when the tumors reach tumor end point (1500 mm³) or the mice show greater than 15% weight loss. A result showing that a slower tumor growth in the candidate treated group as compared to controls, or a longer mean time to reach the tumor end point volume (1500 mm³) is an indication that the candidate has activity in inhibiting cancer growth.

### Combination Therapy

The method disclosed herein involves a combination therapy of administering both a SGRM and a checkpoint inhibitor to a subject that suffers from a tumor load, which, in some cases, is due to the presence of a checkpoint-inhibitor-sensitive cancer. In some embodiments, the method disclosed herein involves a combination therapy of administering both a SGRM and a checkpoint inhibitor to a subject that suffers from a tumor load of a tumor type that is not traditionally considered a checkpoint-inhibitor-sensitive cancer, but that may be induced to become sensitive to a checkpoint inhibitor with GRM or SGRM administration. In some embodiments, the combination therapy involves administration of a checkpoint inhibitor and a SGRM sequentially in any order during the entire or portions of the treatment period.

In some cases, the SGRM and the checkpoint inhibitor are administered following the same or different dosing regimen. For example, the GRM or SGRM may be administered alone for a day, or two days, or three days, or a week, or other lead-in period, and then the checkpoint inhibitor may be administered following such initial GRM or SGRM lead-in period. In some cases, the SGRM is administered following a scheduled regimen while the checkpoint inhibitor is administered intermittently. In some cases, the checkpoint inhibitor is administered following a scheduled regimen while the SGRM is administered intermittently. In some cases, both the SGRM and the checkpoint inhibitor are administered intermittently. In some embodiments, the SGRM is administered daily, and the checkpoint inhibitor, e.g., a checkpoint inhibitor, is administered weekly, biweekly, once every three weeks, once every four weeks, or at other intervals. In some embodiments, the SGRM is administered daily for a lead-in period of one, two, three, four, five, six, seven, or other number of days, and then the checkpoint inhibitor, e.g., a checkpoint inhibitor, is administered weekly, biweekly, once every three weeks, once every four weeks, or at other intervals. Administration of the GRM or SGRM may continue on a daily or other regular basis during the time of intermittent administration of the checkpoint inhibitor.

In some cases, the SGRM and the checkpoint inhibitor are administered sequentially or simultaneously once or twice per month, three times per month, every other week, once per week, twice per week, three times per week, four times per week, five times per week, six times per week, every other day, daily, twice a day, three times a day or more frequent, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, the combination therapy includes co-administering a SGRM and a checkpoint inhibitor. In some embodiments, co-administration of a checkpoint inhibitor and a SGRM involves administering the two agents simultaneously or approximately simultaneously (e.g., within about 1, 5, 10, 15, 20, or 30 minutes of each other).

### Duration

The duration of treatment with a SGRM and a checkpoint inhibitor to reduce tumor load can vary according to the severity of the condition in a subject and the subject’s response to the combination therapy. In some embodiments, the SGRM and/or the checkpoint inhibitor can be administered for a period of about 1 week to 104 weeks (2 years), more typically about 6 weeks to 80 weeks, most typically about 9 to 60 weeks. Suitable periods of administration also include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 104 weeks. Suitable periods of administration also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, 100, and 104 weeks. Generally, administration of a SGRM and/or a checkpoint inhibitor should be continued until the desired clinically significant reduction or amelioration is observed. Treatment with a SGRM and a checkpoint inhibitor in accordance with the invention may last for as long as two years or even longer. In some embodiments, the duration of the SGRM administration is the same as that of the checkpoint inhibitor. In some embodiments, the duration of SGRM administration is shorter or longer than that of the checkpoint inhibitor.

In some embodiments, administration of a SGRM or a checkpoint inhibitor is not continuous and can be stopped for one or more periods of time, followed by one or more periods of time where administration resumes. Suitable periods where administration stops include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 100 weeks. Suitable periods where administration stops also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, and 100 weeks.

### Evaluate Improvements in Reducing Tumor Loads

The combination therapy disclosed herein can reduce tumor load. Methods for measuring these responses are well-known to skilled artisans in the field of cancer therapy, e.g., as described in the Response Evaluation Criteria in Solid Tumors (“RECIST”) guidelines, available at http://ctep.cancer.gov/protocolDevelopment/docs/recist_guideline.pdf.

In one approach, the tumor load is measured by assaying expression of tumor-specific genetic markers. This approach is especially useful for metastatic tumors or tumors that are not easily measurable, e.g., bone marrow cancer. A tumor-specific genetic marker is a protein or other molecule that is unique to cancer cells or is much more abundant in them as compared to non-cancer cells. For example, see WO 2006104474. Non-limiting examples of tumor-specific genetic markers include, alpha-fetoprotein (AFP) for liver cancer, beta-2-microglobulin (B2M) for multiple myeloma; beta-human chorionic gonadotropin (beta-hCG) for choriocarcinoma and germ cell tumors; CA19-9 for pancreatic cancer, gall bladder cancer, bile duct cancer, and gastric cancer; CA-125 and HE4 for ovarian cancer; carcinoembryonic antigen (CEA) for colorectal cancer; chromogranin A (CgA) for neuroendocrine tumor; fibrin/fibrinogen for bladder cancer; prostate-specific antigen (PSA) for prostate cancer; and thyroglobulin for thyroid cancer. See, http://www.cancer.gov/about-cancer/diagnosis-staging/diagnosis/tumor-markers-fact-sheet.

Methods of measuring the expression levels of a tumor-specific genetic marker are well known. In some embodiments, mRNA of the genentic marker is isolated from the blood sample or a tumor tissue and real-time reverse transcriptase-polymerase chain reaction (RT-PCR) is performed to quantify expression of the genetic marker. In some embodiments, western blots or immunohistochemistry analysis are performed to evaluate the protein expression of the tumor-specific genetic marker. Typically the levels of the tumor-specific genetic marker are measured in multiple samples taken over time of the combination therapy of the invention, and a decrease in levels correlates with a reduction in tumor load.

In another approach, the reduction of tumor load by the combination therapy disclosed herein is shown by a reduction in tumor size or a reduction of amount of cancer in the body. Measuring tumor size is typically achieved by imaging-based techniques. For example, computed tomography (CT) scan can provide accurate and reliable anatomic information about not only tumor shrinkage or growth but also progression of disease by identifying either growth in existing lesions or the development of new lesions or tumor metastasis.

In another approach, a reduction of tumor load can be assessed by functional and metabolic imaging techniques. These techniques can provide earlier assessment of therapy response by observing alterations in perfusion, oxygenation and metabolism. For example, ¹⁸F-FDG PET uses radiolabeled glucose analogue molecules to assess tissue metabolism. Tumors typically have an elevated uptake of glucose, a change in value corresponding to a decrease in tumor tissue metabolism indicates a reduction in tumor load. Similar imaging techniques are disclosed in Kang et al., Korean J. Radiol. (2012) 13(4) 371-390.

A patient receiving the combination therapy disclosed herein may exhibit varying degrees of tumor load reduction. In some cases, a patient can exhibit a Complete Response (CR), also referred to as “no evidence of disease (NED)”. CR means all detectable tumor has disappeared as indicated by tests, physical exams and scans. In some cases, a patient receiving the combination therapy disclosed herein can experience a Partial Response (PR), which roughly corresponds to at least a 50% decrease in the total tumor volume but with evidence of some residual disease still remaining. In some cases the residual disease in a deep partial response may actually be dead tumor or scar so that a few patients classified as having a PR may actually have a CR. Also many patients who show shrinkage during treatment show further shrinkage with continued treatment and may achieve a CR. In some cases, a patient receiving the combination therapy can experience a Minor Response (MR), which roughtly means a small amount of shrinkage that is more than 25% of total tumor volume but less than the 50% that would make it a PR. In some cases, a patient receiving the combination therapy can exhibit Stable Disease (SD), which means the tumors stay roughly the same size, but can include either a small amount of growth (typically less than 20 or 25%) or a small amount of shrinkage (Anything less than a PR unless minor responses are broken out. If so, then SD is defined as typically less 25%).

Desired beneficial or desired clinical results from the combination therapy may also include e. g., reduced (i.e., slowing to some extent and/or stop) cancer cell infiltration into peripheral organs; inhibit (i.e., slow to some extent and/or stop) tumor metastasis; increased response rates (RR); increased duration of response; relieved to some extent one or more of the symptoms associated with the cancer; decreased dose of other medications required to treat the disease; delayed progression of the disease; and/or prolonged survival of patients and/or improved quality of life. Methods for evaluating these effects are well known and/or disclosed in, e.g., http://cancerguide.org/endpoints.html and RECIST guidelines, supra.

### Pharmaceutical Compositions and Administration

GRMs and SGRMs (as used herein, GRMs and SGRMs include nonsteroidal GRMs and nonsteroidal SGRMS), can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. GRMs and SGRMs can also be administered by injection, that is, intravenously, intramuscularly, intracutaneously, subcutaneously, intraduodenally, or intraperitoneally. Also, GRMs and SGRMs can be administered by inhalation, for example, intranasally. Additionally, GRMs and SGRMs can be administered transdermally. Accordingly, the present invention also provides pharmaceutical compositions including a pharmaceutically acceptable carrier or excipient and a GRM or SGRM.

For preparing pharmaceutical compositions from GRMs and SGRMs, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington’s Pharmaceutical Sciences, Mack Publishing Co, Easton PA (“Remington’s”).

In powders, the carrier is a finely divided solid, which is in a mixture with the finely divided active component, a GRM or SGRM. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethylcellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain GR modulator mixed with a filler or binders such as lactose or starches, lubricants such as talc or magnesium stearate, and, optionally, stabilizers. In soft capsules, the GR modulator compounds may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending a SGRM in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

GRMs and SGRMs can be delivered by transdermally, by a topical route, formulated as applicator sticks, solutions, suspensions, emulsions, gels, creams, ointments, pastes, jellies, paints, powders, and aerosols.

GRMs and SGRMs can also be delivered as microspheres for slow release in the body. For example, microspheres can be administered via intradermal injection of drug -containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use

In another embodiment, the formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component, a GRM or SGRM. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 6000 mg, most typically 50 mg to 500 mg. Suitable dosages also include about 1 mg, 5, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 mg, according to the particular application and the potency of the active component. The composition can, if desired, also contain other compatible therapeutic agents.

The pharmaceutical preparation is preferably in unit dosage form. In such form the preparation is subdivided into unit doses containing appropriate quantities of the compounds and compositions of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

GRMs can be administered orally. For example, the GRM can be administered as a pill, a capsule, or liquid formulation as described herein. Alternatively, GRMs can be provided via parenteral administration. For example, the GRM can be administered intravenously (e.g., by injection or infusion). Additional methods of administration of the compounds described herein, and pharmaceutical compositions or formulations thereof, are described herein.

In some embodiments, the GRM is administered in one dose. In other embodiments, the GRM is administered in more than one dose, e.g., 2 doses, 3 doses, 4 doses, 5 doses, 6 doses, 7 doses, or more. In some cases, the doses are of an equivalent amount. In other cases, the doses are of different amounts. The doses can increase or taper over the duration of administration. The amount will vary according to, for example, the GRM properties and patient characteristics.

Any suitable GRM dose may be used in the methods disclosed herein. The dose of GRM that is administered can be at least about 300 milligrams (mg) per day, or about 600 mg/ day, e.g., about 600 mg/day, about 700 mg/day, about 800 mg/day, about 900 mg/day, about 1000 mg/day, about 1100 mg/day, about 1200 mg/day, or more. For example, where the GRA is mifepristone, the GRM dose may be, e.g., 300 mg/day, or 600 mf/ day, or 900 mg/day, or 1200 mg/day of mifepristone. In embodiments, the GRM is administered orally. In some embodiments, the GRM is administered in at least one dose. In other words, the GRM can be administered in 1, 2, 3, 4, 5, 6, 7, 8, 9, 10 or more doses. In embodiments, the GRM is administered orally in 1, 2, 3, 4, 5, 6, 7, 8, 9, 10 or more doses.

The patient may be administered at least one dose of GRM in one or more doses over, for example, a 2-48 hour period. In some embodiments, the GRM is administered as a single dose. In other embodiments, the GRM is administered in more than one dose, e.g. 2 doses, 3 doses, 4 doses, 5 doses, or more doses over a 2-48 hour period, e.g., a 2 hour period, a 3 hour period, a 4 hour period, a 5 hour period, a 6 hour period, a 7 hour period, a 8 hour period, a 9 hour period, a 10 hour period, a 11 hour period, a 12 hour period, a 14 hour period, a 16 hour period, a 18 hour period, a 20 hour period, a 22 hour period, a 24 hour period, a 26 hour period, a 28 hour period, a 30 hour period, a 32 hour period, a 34 hour period, a 36 hour period, a 38 hour period, a 40 hour period, a 42 hour period, a 44 hour period, a 46 hour period or a 48 hour period. In some embodiments, the GRM is administered over 2-48 hours, 2-36 hours, 2-24 hours, 2-12 hours, 2-8 hours, 8-12 hours, 8-24 hours, 8-36 hours, 8-48 hours, 9-36 hours, 9-24 hours, 9-20 hours, 9-12 hours, 12-48 hours, 12-36 hours, 12-24 hours, 18-48 hours, 18-36 hours, 18-24 hours, 24-36 hours, 24-48 hours, 36-48 hours, or 42-48 hours.

Single or multiple administrations of formulations can be administered depending on the dosage and frequency as required and tolerated by the patient. The formulations should provide a sufficient quantity of active agent to effectively treat the disease state. Thus, in one embodiment, the pharmaceutical formulation for oral administration of a GRM is in a daily amount of between about 0.01 to about 150 mg per kilogram of body weight per day (mg/kg/day). In some embodiments, the daily amount is from about 1.0 to 100 mg/kg/day, 5 to 50 mg/kg/day, 10 to 30 mg/kg/day, and 10 to 20 mg/kg/day. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing parenterally administrable formulations will be known or apparent to those skilled in the art and are described in more detail in such publications as Remington’s, supra. See also Nieman, In “Receptor Mediated Antisteroid Action,” Agarwal, et al., eds., De Gruyter, New York (1987).

The duration of treatment with a GRM or SGRM can vary according to the severity of the condition in a subject and the subject’s response to GRMs or SGRMs. In some embodiments, GRMs and SGRMs can be administered for a period of about 1 week to 104 weeks (2 years), more typically about 6 weeks to 80 weeks, most typically about 9 to 60 weeks. Suitable periods of administration also include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 104 weeks. Suitable periods of administration also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, 100, and 104 weeks. Generally administration of a GRM or SGRM should be continued until clinically significant reduction or amelioration is observed. Treatment with the GRM or SGRM in accordance with the invention may last for as long as two years or even longer.

In some embodiments, administration of a GRM or SGRM is not continuous and can be stopped for one or more periods of time, followed by one or more periods of time where administration resumes. Suitable periods where administration stops include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 100 weeks. Suitable periods where administration stops also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, and 100 weeks.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington’s, supra). The state of the art allows the clinician to determine the dosage regimen for each individual patient, GR modulator and disease or condition treated.

SGRMs can be used in combination with other active agents known to be useful in modulating a glucocorticoid receptor, or with adjunctive agents that may not be effective alone, but may contribute to the efficacy of the active agent.

In some embodiments, co-administration includes administering one active agent, a GRM or SGRM, within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

After a pharmaceutical composition including a GR modulator of the invention has been formulated in an acceptable carrier, it can be placed in an appropriate container and labeled for treatment of an indicated condition. For administration of a GRM or SGRM, such labeling would include, e.g., instructions concerning the amount, frequency and method of administration.

The pharmaceutical compositions of the present invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compositions of the present invention are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer’s solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient’s needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic, parenterally acceptable diluent or solvent, such as a solution of 1,3-butanediol.

### Combination Therapies

Various combinations with a GRM or SGRM and a chemotherapeutic agent, checkpoint inhibitor, or other treatment (e.g., a cancer treatment), or a combination of such agents and compounds, may be employed to treat the patient. By “combination therapy” or “in combination with”, it is not intended to imply that the therapeutic agents must be administered at the same time and/or formulated for delivery together, although these methods of delivery are within the scope described herein. The GRM or SGRM and the chemotherapeutic or other agent can be administered following the same or different dosing regimen. In some embodiments, the GRM or SGRM and the chemotherapeutic or other agent is administered sequentially in any order during the entire or portions of the treatment period. In some embodiments, the GRM or SGRM and the chemotherapeutic or other agent is administered simultaneously or approximately simultaneously (e.g., within about 1, 5, 10, 15, 20, or 30 minutes of each other). Non-limiting examples of combination therapies are as follows, with administration of the GRM or SGRM and the chemo agent for example, GRM or SGRM is “A” and the chemotherapeutic or other agent, given as part of a therapy regime, is “B″:
A/B/AB/A/BB/B/AA/A/BA/B/BB/A/AA/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A
AAA (B/A AAAAAAAAAAAAAAAAAAAA)ₙ
(where the “n” indicates that the cycle enclosed in patentheses may be repeatedat the discretion of the physucian).

Administration of the therapeutic compounds or agents to a patient will follow general protocols for the administration of such compounds, taking into account the toxicity, if any, of the therapy. Surgical intervention may also be applied in combination with the descirbed therapy.

The present methods can be combined with other means of treatment such as surgery, radiation, targeted therapy, immunotherapy, use of growth factor inhibitors, or anti-angiogenesis factors.

All patents, patent publications, publications, and patent applications cited in this specification are hereby incorporated by reference herein in their entireties as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill will readily recognize a variety of noncritical parameters which could be changed or modified to yield essentially similar results.

### Example 1. Hepg2 Tyrosine Aminotransferase (TAT) Assay

The following protocol describes an assay for measuring induction of TAT by dexamethasone in HepG2 cells (a human liver hepatocellular carcinoma cell line; ECACC, UK). HepG2 cells are cultured using MEME media supplemented with 10% (v/v) foetal bovine serum; 2 mM L-glutamine and 1% (v/v) NEAA at 37° C., 5%/95% (v/v) CO₂/air. The HepG2 cells are then be counted and adjusted to yield a density of 0.125 × 10⁶ cells/ml in RPMI 1640 without phenol red, 10% (v/v) charcoal stripped FBS, 2 mM L-glutamine and seeded at 25,000 cells/well in 200 µl into 96 well, sterile, tissue culture micro titre plates, and incubated at 37° C., 5% CO₂ for 24 hours.

Growth media are then removed and replaced with assay media {RPMI 1640 without phenol red, 2 mM L-glutamine + 10 µM forskolin}. Test compounds are then screened against a challenge of 100 nM dexamethasone. Compounds are then be serially half log diluted in 100% (v/v) dimethylsulfoxide from a 10 mM stock. Then an 8-point half-log dilution curve are generated followed by a 1:100 dilution into assay media to give a 10x final assay of the compound concentration, this results in final assay of the compound concentration that ranged 10 to 0.003 µM in 0.1% (v/v) dimethylsulfoxide.

Test compounds are pre-incubated with cells in micro-titre plates for 30 minutes at 37° C., 5/95 (v/v) CO₂/air, before the addition of 100 nM dexamethasone and then subsequently for 20 hours to allow optimal TAT induction.

HepG2 cells are then lysed with 30 µl of cell lysis buffer containing a protease inhibitor cocktail for 15 minutes at 4° C. 155 µl of substrate mixture can then be added containing 5.4 mM Tyrosine sodium salt, 10.8 mM alpha ketoglutarate and 0.06 mM pyridoxal 5′ phosphate in 0.1 M potassium phosphate buffer (pH 7.4). After 2 hours incubation at 37° C. the reaction can be terminated by the addition of 15 µl of 10 M aqueous potassium hydroxide solution, and the plates incubated for a further 30 minutes at 37° C. The TAT activity product can be measured by absorbance at λ 340 nm.

IC₅₀ values can be calculated by plotting % inhibition (normalised to 100 nM dexamethasone TAT stimulation) v. compound concentration and fitting the data to a 4 parameter logistic equation. IC₅₀ values can converted to Ki (equilibrium dissociation constant) using the Cheng and Prusoff equation, assuming the antagonists were competitive inhibitors with respect to dexamethasone.

### Example 2. Relacorilant Stimulates Anti-Tumor Immune Response

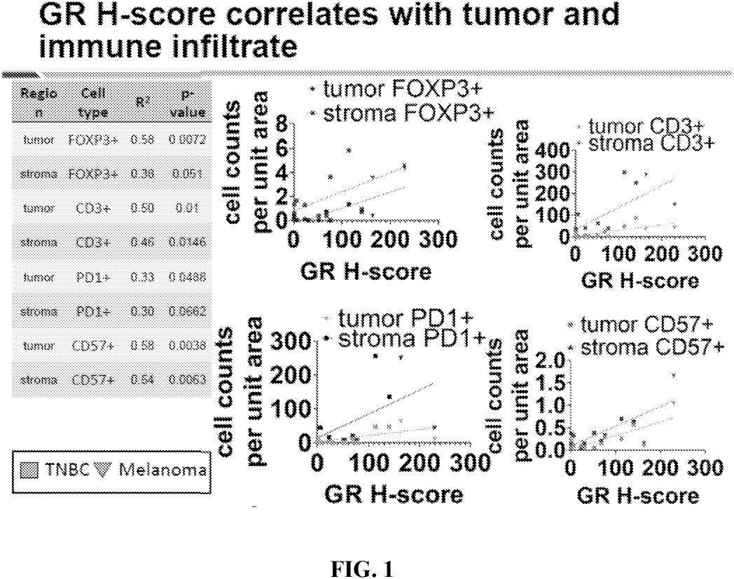

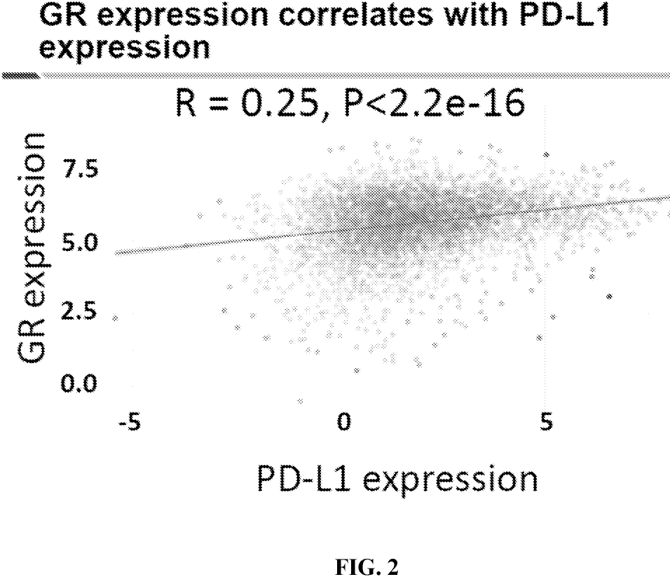

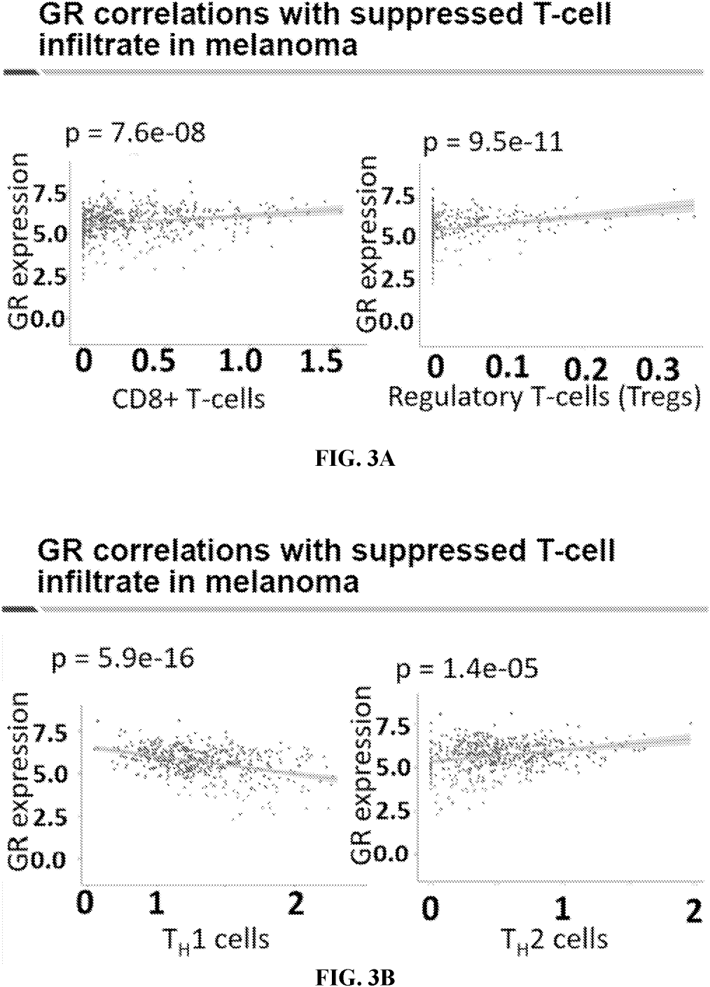

Response to an immune checkpoint inhibitor (ICI) is associated with tumor immune infiltration and PD-L1 expression, so we first assessed whether GR expression was observed in the same types of tumors likely to respond to ICI. In melanoma and TNBC tumors, CD3+ T-cell infiltration correlated with GR expression (FIG. 1). GR expression also correlated with FOXP3+ cells, a marker of T_{regs} that suppress cytotoxic T-cell function. Analysis of transcript data from the National Cancer Institute’s The Cancer Genome Atlas (TCGA; accessible at the National Cancer Institute “cancer.gov” website at page “about-nci/organization/ccg/research/structural-genomics/tcga”) showed that GR expression correlated with markers of immunosuppressive cells. A global correlation of GR and PDL1 was observed (p < 2×10⁻¹⁶), with particularly high correlations in adrenal, bladder, and pancreatic cancers. FIG. 2 shows that GR expression correlates with PD-L1 expression. Using xCell (Aran, Genome Biology 2017) to estimate abundance of distinct immune cell types within individual tumors, a positive correlation between GR and CD8+ T-cells, Tregs, and Th₂ cells was observed. FIG. 3A shows that GR expression positively correlates with CD8+ T-cells and regulatory T-cells (Tregs)). FIG. 3B shows that GR expression negatively correlates T_{H}1 T-cells and positively correlates with T_{H}2 T-cells. Tregs are believed to limit the ability of CD8+ T-cells to activate and eliminate tumors. These data suggest that GR is elevated in tumors with suppressed T-cell infiltrate, a class of tumors that are generally considered good candidates for ICI therapy.

### Cortisol Suppresses Activation of Human PBMC’s and Activation Is Restored by Relacorilant

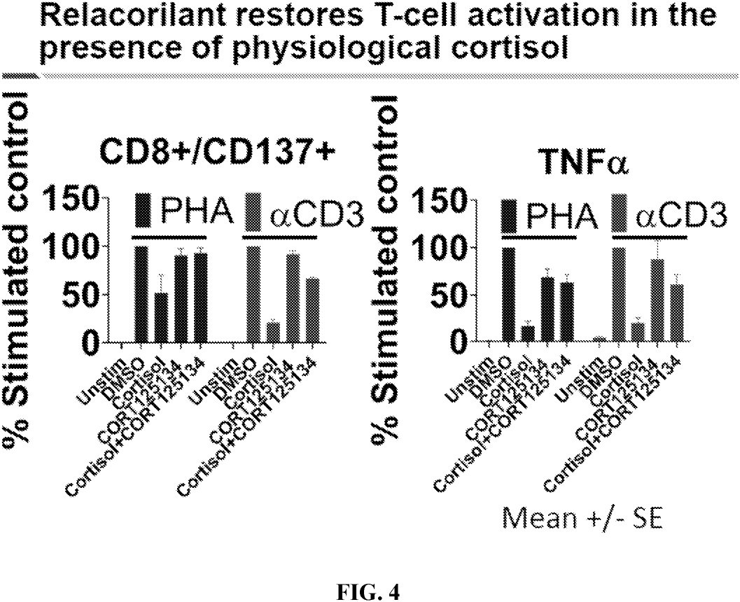

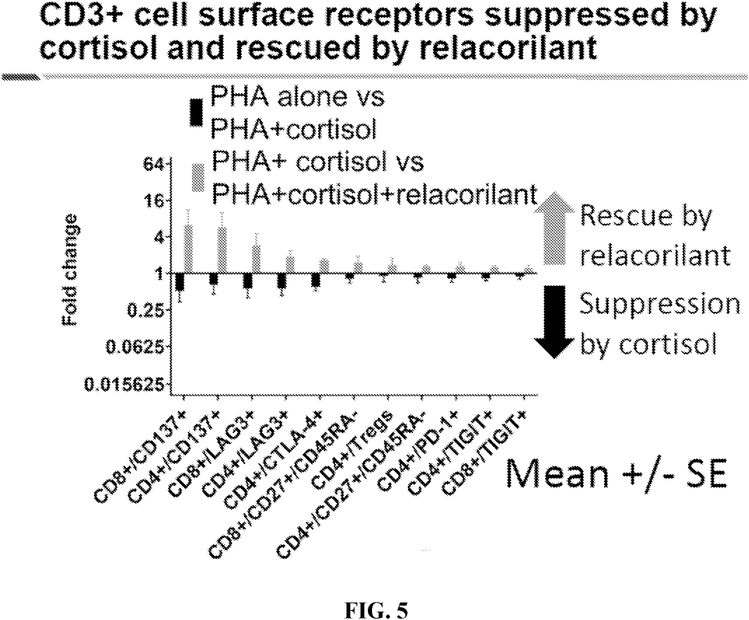

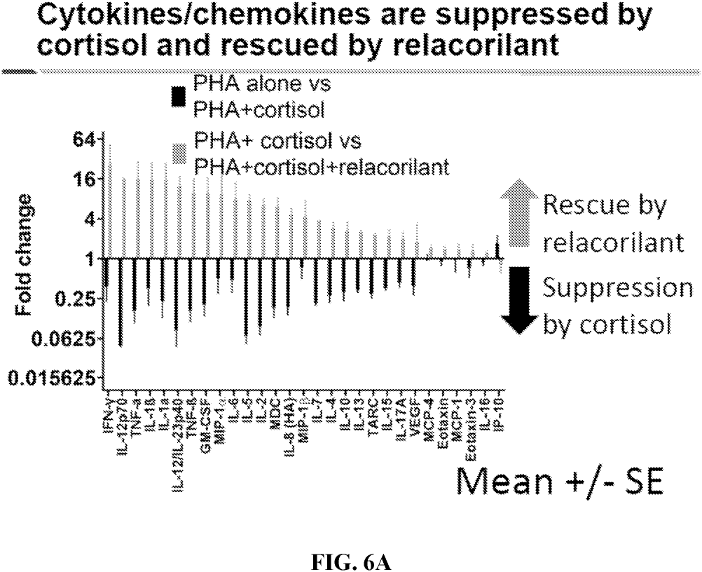

To understand the molecular consequences of GC activity on T-cell activation, the effects of cortisol and relacorilant were assessed on stimulated human PBMC’s. 400 nM cortisol, a concentration typically found in human serum, potently suppressed nearly every phenotypic effect of stimulation by either phytohemagglutinin (PHA) or αCD3+IL-12. Expression of CD137 (aka 41-BB) on CD8+ cells was reduced by cortisol and rescued by relacorilant. FIG. 4 shows the restoration of T-cell activation by relacorilant in the presence of physiological levels of cortisol. Expression of CD137 (aka 41-BB) within CD8+ cells was reduced by cortisol and rescued by relacorilant. A similar trend was observed for other T-cell subsets that were stimulated by PHA or αCD3+IL-12 (FIG. 5 and FIG. 6), including CD8+ and CD4+ expressing LAG3 and CTLA4. FIG. 5 shows, following stimulation by phytohemagglutinin (PHA), suppression of CD3+ cell surface receptors by cortisol, and the restoration of the CD3+ cell surface receptors by relacorilant. Thus, as shown also in FIG. 4, inflammatory cytokines such as TNF-α were induced by stimulation, suppressed by cortisol, and rescued by relacorilant. A similar pattern was observed for cytokines and chemokines induced by stimulation (FIG. 6A and FIG. 6B), including IFNγ, IL-1β, IL-1α, and IL-6. FIG. 6A and FIG. 6B show, following stimulation by phytohemagglutinin (PHA) (FIG. 6A) or αCD3 (FIG. 6B), suppression of cytokines and chemokines by cortisol and the restoration of cytokine/chemokine levels by relacorilant. (Supernatant IL-12 measurements were excluded from the analysis shown in FIG. 6B since the stimulation included recombinant IL-12.) Physiological levels of cortisol suppressed cytokines and chemokines, and this suppression was reversed by relacorilant. These results demonstrate a broad immunosuppressive effect on T-cell activation mediated by cortisol at normal physiological concentrations, and these effects were reversed by relacorilant.

### Relacorilant Promotes T-Cell Function and aPDl Response in a Syngeneic Mouse Model

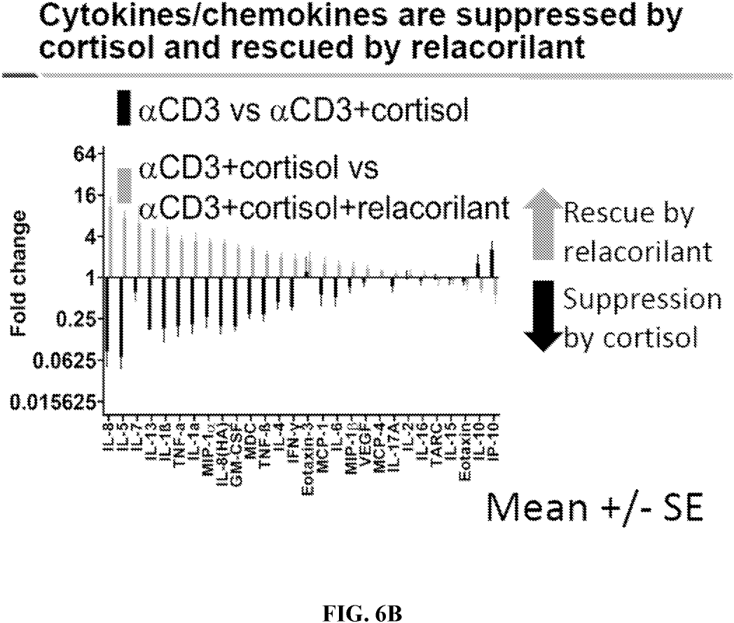

The suppressive effects of cortisol on CD8+ cytotoxic T-cells, and the ability of relacorilant to promote T-cell activation, were assessed in the EG7 syngeneic mouse model. EG7 tumor cells express ovalbumin, and the model was studied both in WT or OT-1/Rag^{-/-} mice. The OT-1/Rag^{-/-} mice only have T-cells expressing a transgenic ovalbumin-specific TCR. In the OT-1/Rag^{-/-} background, untreated mice were able to control tumor growth for 17-20 days (FIG. 7). The combination of PD1 antagonist antibody (RMP1-14) and relacorilant was assessed in the EG7 tumor model. Relacorilant significantly increased the efficacy of an anti-PD1 antibody in this model. Because mice do not synthesize cortisol at levels equivalent to humans, cortisol was administered in the drinking water at 100 mg/L which resulted in average serum cortisol levels of 447 nM (data not shown). Cortisol administration resulted in rapid tumor growth (FIG. 7). Premature deaths occurred in 2/5 mice treated with cortisol and 0/5 control mice. All mice treated with cortisol had measurable tumors by day 10, while 2/5 control mice had no detectable tumor from days 10-20. When the OT-1/Rag^{-/-} mice were given cortisol +/- relacorilant, 2/7 histologically confirmed complete remissions were observed in the cortisol+relacorilant-treated group while none of the cortisol-alone group had remission. In contrast, administration of cortisol to the drinking water of wild type (WT) mice had no effect on tumor control or growth (data not shown). Together, these data suggest that cortisol suppresses tumor elimination by cytotoxic CD8+ T-cells and relacorilant restores cytotoxic CD8+ T-cell function.

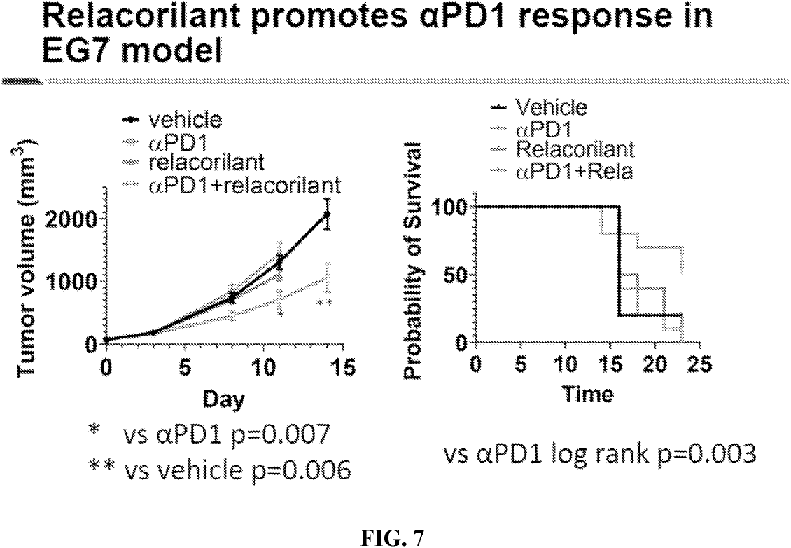

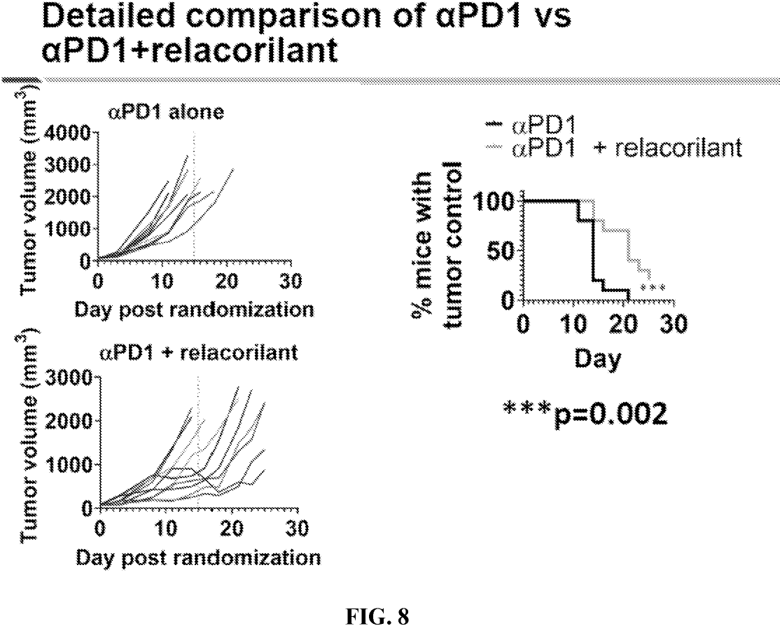

The combination of PD1 antagonist antibody (RMP1-14) and relacorilant was assessed in the EG7 tumor model. Most reports have assessed αPD1 effects on EG7 cells in WT mice without added cortisol, so this more established model was used. Relacorilant or αPD1 alone had no significant effect in this model. The combination of relacorilant and αPD1 suppressed tumor growth (FIG. 8). By day 14, 8/10 mice in the αPD1 alone arm had tumors larger than 1800 mm3, compared to 2/10 in the αPD1+relacorilant groups. Time to ethical sacrifice or 1800 mm³ was also significantly better in the relacorilant + αPD1 group as compared to the αPD1 group alone (FIG. 8). Assessment of the individual mouse tumor volume trajectories show significant control between days 10-20 of this aggressive model. Excess cortisol administration reversed the effects of relacorilant and restored tumor growth, demonstrating that the relacorilant effects are specific to antagonism of cortisol activity. Terminal sera collected between days 11 and 21 of the study showed that TNFα levels were increased by the addition or relacorilant but suppressed by the addition of cortisol. Consistent with the effects observed in isolated human peripheral blood mononuclear cells (PBMCs), the ability of relacorilant to promote T-cell function and pro-inflammatory cytokine secretion is recapitulated in this model.

### Systemic Effects of Relacorilant in a Phase I Study in Solid Tumor Patients Demonstrate Antagonism of Endogenous GR Activity

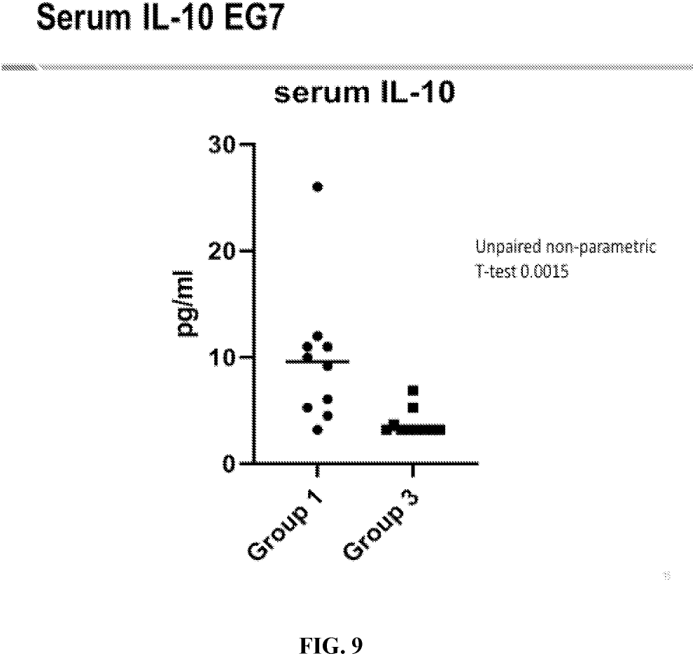

GR is a broad regulator of immunosuppressive transcriptional programs, so we first assessed the transcriptional effects of prednisone in and/or relacorilant in whole blood. In a healthy volunteer phase I study, a 25 mg dose of prednisone resulted in a large transcriptional effect 4 hours post dose. This defined a gene set of prednisone-induced genes in whole blood. In a phase I study of relacorilant+nab-paclitaxel in solid tumor patients, the prednisone-induced genes were predominantly suppressed. A significant overlap in the two gene sets was observed only in patients that benefited from therapy, as a defined by a RECIST best overall response of SD or better. In patients with progressive disease, there was no significant overlap between genes induced by prednisone and suppressed after dosing with relacorilant+nab-paclitaxel. FIG. 10 shows that combined relacorilant + nab paclitaxel treatment suppressed gene expression in patients with solid tumors. Suppressed genes included genes expressing IL8 (CXCL8), IDO1, and EP4 (PTGER4) (n=46). The neutrophil-to-lymphocyte ratio (NLR) was also normalized in these patients (p=0.01). Canonical GR regulated genes dusp1 and ptgs2 (COX2) were suppressed in patients administered relacorilant+nab-paclitaxel. Among the most suppressed genes after treatment with relacorilant and nab-paclitaxel were cxcl8 (IL-8), idol, and ptger4 (EP4). The reduction in cxcl8 transcript resulted in post-therapy readings below the limit of quantification. These three genes are known to play a role in suppressing the cytotoxic T-cell response. The overall transcriptional effects of relacorilant in whole blood are both reciprocal to the prednisone effects and characteristic of processes that would be expected to promote a productive cytotoxic T-cell response.

GR activity has been shown to alter the cellular composition of blood, so we assessed the effects of relacorilant on neutrophil and lymphocyte abundance. The baseline neutrophil-to-lymphocyte ratio is predictive of response to checkpoint inhibitors, and reduction of the NLR is associated with improved outcomes as well (Lalani et al. Journal for ImmunoTherapy of Cancer (2018) 6:5). First, we established that relacorilant does not affect NLR in healthy volunteers with normal cortisol levels. In healthy volunteers, prednisone resulted in a rapid an acute increase in the NLR. This effect was reversed when relacorilant was co-dosed with prednisone. These data establish that relacorilant does not affect NLR in healthy individuals (under conditions where stress or disease state are not expected to elevate cortisol levels) and that relacorilant can reverse the effects of glucocorticoid agonism on the NLR. In patients with advanced solid tumors, we observed that baseline NLR was higher than healthy subjects. There was an overall decrease in the NLR in the first 8 or 15 days in all patients. This decrease was pronounced in patients with baseline NLR elevation (NLR >3), but no significant change in NLR was observed in patients with normal NLR at baseline (NLR ≤3). The decrease in NLR in the first 15 days of therapy was correlated with the Cₘₐₓ of relacorilant but not paclitaxel, suggesting the effects are primarily driven by GR antagonism. There was a non-significant trend toward more pronounced clinical benefit in patients with a decrease in NLR. These data demonstrate that NLR is increased by GR agonist and decreased by GR antagonist.

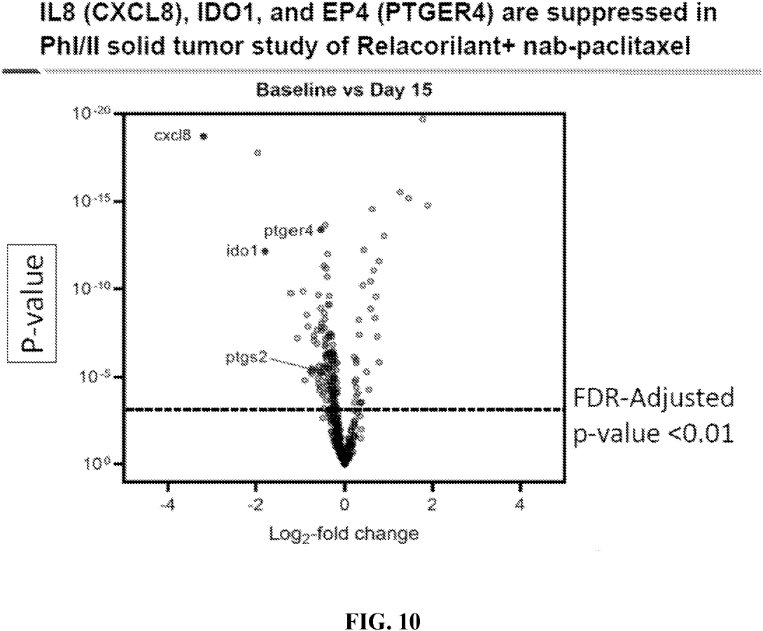

In the small phase I solid tumor study, one patient achieved a complete response per RECIST 1.1 after treatment with relacorilant+nab-paclitaxel. This observation was unexpected given the patients history and prior lines of treatment. FIG. 11 shows a summary of effects on selected biomarkers in a patient with complete response (CR) to treatment with relacorilant + nab-paclitaxel. This patient exhibited a decrease in neutrophil-to-lymphocyte ratio (NLR), and changes in CD4+ cells, CD8+ cells, CD3+ T-cells, expression of ptgs2 and dusp1 and other changes. (C1D1 indicates cycle 1 day 1 of treatment; C1D15 indicates cycle 1 day 15 of treatment; C4D1 indicates cycle 4 day 1 of treatment, and EOT indicates end of treatment.) In this patient, the NLR declined from 5.5 (elevated) to 2.5 (normal) after 8 days of therapy (upper left of FIG. 11). This NLR improvement was accompanied by a reduction in GR-controlled transcripts ptgs2 and dusp1 (lower left of FIG. 11). The abundance of these transcripts rebounded to above baseline as the disease later progressed, treatment with relacorilant was discontinued, and dexamethasone was eventually administered. A decrease in T_{reg}’s (as a % of CD4+ T-cells) and in increase in CD3+ (as a % of mononuclear CD45+), CD4+ (as a % of CD3+), and CD8+ (as % of CD3+) was observed (upper right of FIG. 11). Plasma IFN-γ slightly increased while IL-10 decreased in this patient (lower right of FIG. 11). These observations are consistent with immune activation and antagonism of cortisol activity.

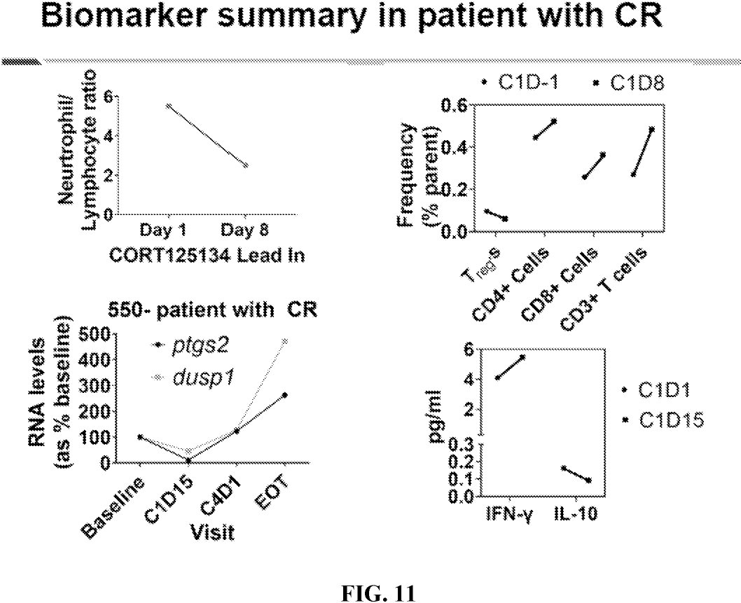

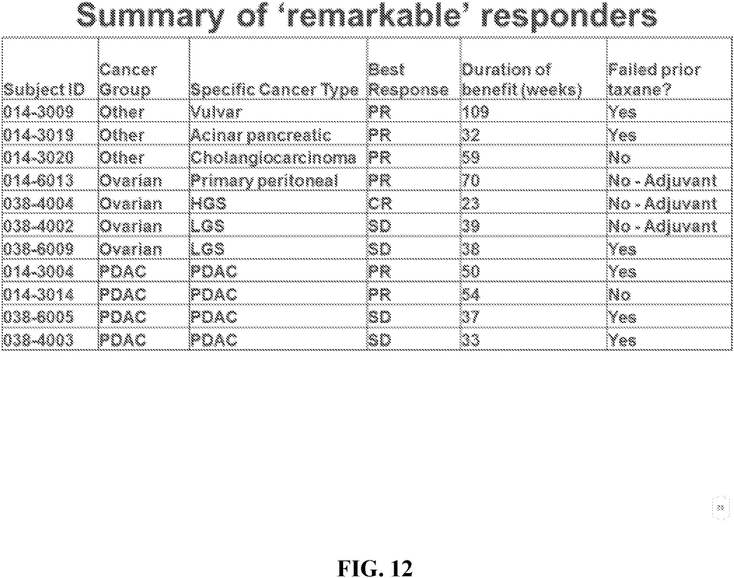

Based on this observation, immune responses in other patients with long duration of response to relacorilant+nab-paclitaxel was assessed. As is common in ICI trials, a small group (10 of 57 evaluable patients) had a sustained benefit (FIG. 12). This was particularly surprising given their disease state and, in some cases, prior duration of response to nab-paclitaxel therapy (FIG. 12). These patients had an increase in circulating CD3+ cells and plasma IFNγ levels. This was accompanied by a decrease in in circulating T_{reg}s, plasma IL-10 levels, and transcription of GR-controlled genes in whole blood (FIG. 13).

As shown in FIG. 13, there is evidence of immune activity in patients with unusually durable responses on relacorilant + nab-paclitaxel. These patients exhibited these trends in plasma/whole blood: decreased NLR (D8 p = 0.006; D15 p = 0.02); decreased numbers of T_{reg}s (p = 0.06); increased numbers of CD3+ cells (p = 0.06); decreased GR-controlled gene expression (ptgs2) in whole blood (p = 0.008) early, rebound at EOT; increased IFNγ (p = 0.03 (excluding a high outlier)); and decreased IL-10 (p=0.03), among the trends found in such patients. These trends were not observed across the broader trial population. Additionally, the NLR in these remarkable responders decreased from bassline to C1D8 and C1D15 (FIG. 13). Together, these observations suggest the long duration of benefit was associated with an immune response to relacorilant + nab-paclitaxel.

### Conclusions

Relacorilant is a potent and selective GR antagonist with demonstrated systemic GR antagonism in healthy volunteers and patients with advanced solid tumors. GR expression is abundant in human tumors and immune cells, and high tumor GR levels are associated with high immune infiltrate and PDL1 expression. Physiological concentrations of cortisol broadly suppress human PBMC activation in vitro, and relacorilant rescues this suppression. Combination of relacorilant with a αPD1 was demonstrated in a syngeneic mouse model, EG7. The systemic effects of relacorilant were consistent with the reciprocal of GR agonist effects in phase I studies in solid tumors patients and healthy volunteers.

Key correlates of response to immune checkpoint inhibitors (ICI) have been defined clinically. Immune infiltration into the tumor (often called “hot” tumors) and PDL1 expression in the tumor tend to predict better responses to checkpoint inhibitors, and GR abundance correlates with both. This suggests an overlapping subset of tumors exists with high GR, immune infiltrate, and PDL1 expression. GR antagonism may re-activate these infiltrated, suppressed immune cells. Induction of pro-inflammatory signals like TNF-α and IFN-γ, in concert with suppression of immunosuppressive signals like IL-8, EP4, and IDO1, have been associated with ICI response. Endogenous cortisol modulates these pathways in a direction expected to reduce ICI response while relacorilant has the reciprocal effect. Low NLR predicts response to checkpoint inhibitor, and relacorilant lowers the NLR in cancer patients with elevated baseline NLR. Thus the effects of relacorilant would likely suppress pathological endogenous cortisol activity and promote ICI responses.

Elevated endogenous cortisol activity has been reported in patients with cancer, and relacorilant data confirms that endogenous cortisol activity can be antagonized. The normalization of NLR by a GR antagonist suggests that elevated NLR in cancer patients may be driven, in part, by elevated cortisol activity. The elevated NLR was not caused by administration of synthetic GR agonist as such therapies were prohibited in the study. Similarly, antagonism of GR-controlled genes by relacorilant in the patients demonstrating a benefit on relacorilant + nab-paclitaxel suggests some endogenous GR-agonist activity was present prior to treatment. Since baseline synthetic steroid use is associated with poor outcomes with ICI, baseline elevated cortisol activity could be responsible for limiting ICI responses in some patients.

### Example 3. Relacorilant Reversal of Cortisol Effects in Solid Tumors

Introduction: Cortisol, an endogenous glucocorticoid receptor (GR) agonist, controls a broad transcriptional program that affects T-cell activation, pro-inflammatory cytokine secretion, and immune cell trafficking. By selectively antagonizing GR, relacorilant may reverse the immunosuppressive effects of cortisol in solid tumor cancers.

Methods: Immune cell abundance and GR expression were assessed by IHC and calculated based on The Cancer Genome Atlas (TCGA) data. Human PBMCs were stimulated with αCD3+IL-12 +/- cortisol or cortisol + relacorilant. EG7 tumor-bearing mice were treated with αPD1 (RMP1-14) ip (intraperitoneally) Q5D (every fifth day) +/- daily relacorilant (QD). Whole blood mRNA was measured via Nanostring, hematology was performed using standard complete blood count assays, and cytokines were assessed by immunoassays in study NCT02762981.

Results: GR expression was observed in human tumor and immune cells. Its abundance was positively correlated with tumor infiltration of T_{H}2, Treg, and PDL1⁺ cells (P<0.001) and negatively correlated with T_{H}1 cells (P<0.001). In PBMCs, cortisol inhibited, and relacorilant restored, CD8⁺ T-cell activation (P<0.001) and pro-inflammatory cytokine secretion (TNFα P=0.006, IFNγ P<0.05). In the EG7 syngeneic model, relacorilant increased αPD1 efficacy (P=0.007) and decreased circulating IL-10 (P<0.002). In patients with advanced solid tumors, relacorilant + nab-paclitaxel systemically suppressed the expression of canonical GR-controlled genes (ptgs2 P<0.001) and genes encoding candidate-immunomodulatory drug targets (cxcl8, ptger4, idol; P<0.001). (FIG. 10, n=46). In a small subset of patients (n=11), sustained clinical response was associated with increased T-cell count (P=0.06) and IFNγ (P=0.03), as well as decreased Tregs. The neutrophil-to-lymphocyte ratio (NLR) was also normalized in these patients (p=0.01)

Conclusions: Evidence of T-cell activation by relacorilant was observed in PBMCs, syngeneic mouse tumors, and patients with sustained response in a Phase 1 study. This supports the hypothesis that relacorilant can reverse immune suppression by endogenous cortisol in solid tumor cancers.

### Example 4. Short-Term Relacorilant Effects on T-Cells

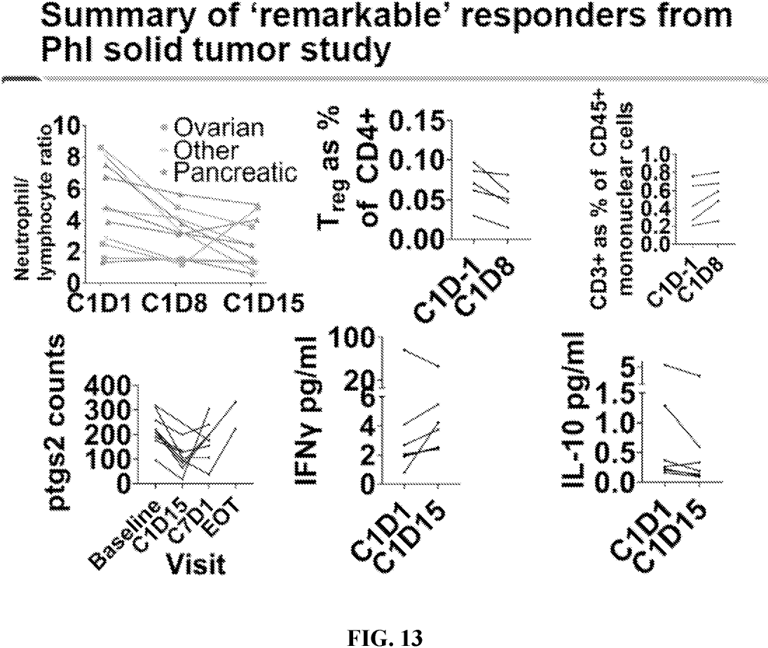

A short term (7-day) EG7 pharmacodynamic study was conducted in female B6 CD45.1 mice to assess the effects of relacorilant+ αPD1 on T-cell proliferation and activation. Spleen and portions of tumor from B6 CD45.1 female mice subcutaneously inoculated with E.G7-OVA mouse lymphoma cells and treated with CORT125134 (30 mg/kg, administered p.o. once daily for 7 days) and RMP1-14 (10 mg/kg, administered i.p. on every fifth day for a total of two doses), alone and in combination, were analyzed via flow cytometry. Unlike the previous study, cell and cytokine analyses were synchronized and occurred before differences in tumor volume were detected (FIG. 14). Thus, in this study, changes in tumor volumes cannot influence the cytokine or T-cell measurements. There were no adverse effects of the treatments on clinical signs or body weight changes.

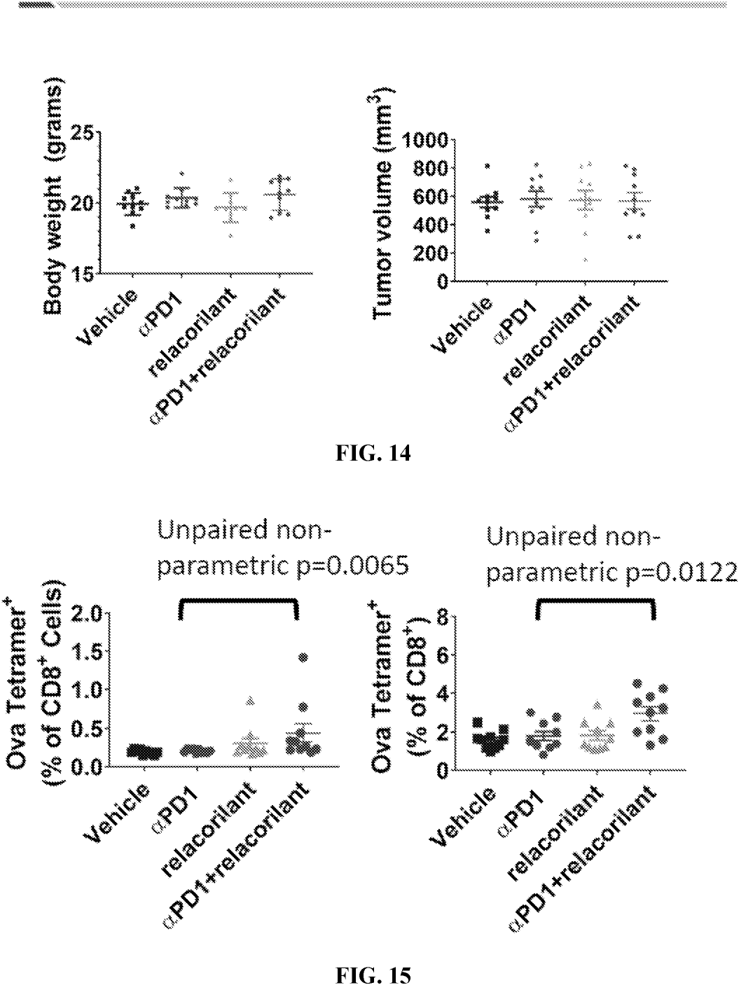

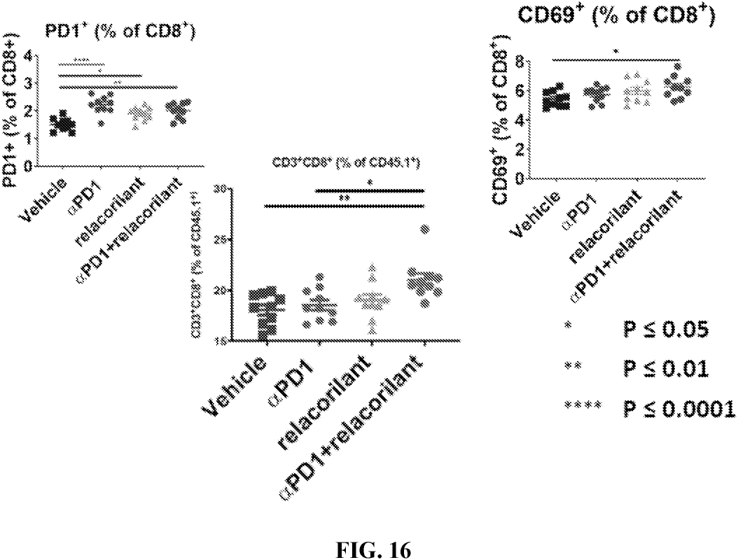

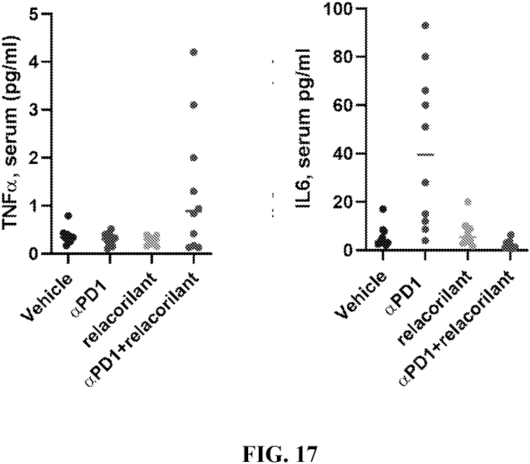

Antigen specific T-cells are key mediators of the anti-tumor immune response. The EG7 model expresses the model antigen ovalbumin. Antigen specific T-cells can be quantified by measuring T-cells that recognize ovalbumin. Cells which bind T-cells markers (such as anti-CD3 and anti-CD8) and bind labeled ovalbumin tetramers are thus considered antigen specific T-cells. Antigen specific T-cells were increased by the combination of relacorilant + αPD1 in the spleen and tumor (FIG. 15). CD69 expression, a marker of T-cell activation, in splenic CD8+ T-cells was increased by the combination as well (FIG. 16). Relacorilant or αPD1 alone was sufficient to induce PD1 expression in splenic CD8-T-cells. (FIG. 16). CD3+CD8+ T-cells were increased in the spleen by the combination (FIG. 16). TNFα in the sera was increased by the combination (FIG. 17). While αPD1 alone raised IL-6 levels, the combination of relacorilant + αPD1 achieved efficacy and expansion of antigen-specific T-cells without raising IL-6 (FIG. 17). The observed in vivo effects, including T-cell activation and TNFα secretion, are consistent with the in vitro effects observed in isolated human PBMC’s.

Conclusions: Administration of relacorilant with αPD1 increased antigen specific T-cell numbers in spleen and tumors in WT mice, and increased CD69 expression in spleen as well. This combination was effective to increase antigen-specific T-cell numbers without raising IL-6. The combination therapy of RMP1-14 / CORT125134 (10 / 30 mg/kg) resulted in a significant (p≤0.05) increase in OVA Tetramer+ as % CD8+ cells in tumors compared with Vehicle Control and the RMP1-14 and CORT125134 monotherapies, and significantly (p≤0.05) higher levels of CD8+OVA Tetramer+ as % of CD3+ cells compared with Vehicle Control. The RMP1-14 and CORT125134 monotherapies and the R MP1-14 / CORT125134 combination therapy resulted in a significant (p≤0.05) increase in PD-1+ as % CD8+ cells in spleens compared with Vehicle Control. The combination therapy also led to significantly (p≤0.05) higher levels of CD3+CD8+ as % of CD45.1+ cells in spleen compared with Vehicle Control and RMP1-14 monotherapy. These effects, including T-cell activation and TNFα secretion, are consistent with the in vitro effects observed in isolated human PBMCs.

All patents, patent publications, publications, and patent applications cited in this specification are hereby incorporated by reference herein in their entireties as if each individual publication or patent application were specifically and individually indicated to be incorporated by reference. In addition, although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of improving immune function in a cancer patient having a solid tumor, the method comprising administering an effective amount of a cancer treatment and an effective amount of a nonsteroidal selective glucocorticoid receptor modulator (SGRM) to said cancer patient,
wherein said improvement in immune function is effective to elicit an anti-cancer effect in said patient having a solid tumor, thereby slowing tumor growth, stopping tumor growth, reducing tumor load, or combinations thereof, and
wherein said improved immune function comprises:
a) increased CD8+ T-cell activation as compared to CD8+ T-cell activation prior to administration of said nonsteroidal SGRM, or
b) increased pro-inflammatory cytokine secretion as compared to pro-inflammatory cytokine secretion prior to administration of said nonsteroidal SGRM, or
c) increased TNFα secretion as compared to TNFα secretion prior to administration of said nonsteroidal SGRM, or
d) increased IFNγ secretion as compared to IFNγ secretion prior to administration of said nonsteroidal SGRM,
or combinations, thereof,
Whereby the patient’s immune function is improved.

2. (canceled)

3. (canceled)

4. (canceled)

5. (canceled)

6. (canceled)

7. The method of claim 1, wherein said immune function is improved after administration of said nonsteroidal SGRM for a number of days selected from 1, 2, 3, 4, 5, 6, 7, 10, 14, or more days.

8. The method of claim 1, wherein said nonsteroidal SGRM is a compound comprising a heteroaryl ketone fused azadecalin structure having the formula: 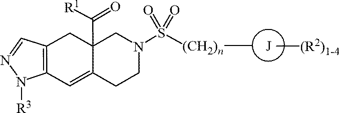
wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁-₆ alkyl, halogen, C₁-₆ haloalkyl, C₁-₆ alkoxy, C₁-₆ haloalkoxy, CN, N-oxide, C₃-₈ cycloalkyl, and C₃-₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁-₆ alkyl, halogen, C₁₆ haloalkyl, C₁₆ alkoxy, C₁-₆ haloalkoxy, C₁-₆ alkyl-C₁-₆ alkoxy, CN, OH, NR^{2a}R^{2b}, C(O)R^{2a} C(O)OR^{2a} C(O)NR^{2a}R^{2b} SR^{2a}, S(O)R^{2a}, S(O)₂R^{2a}, C₃-₈ cycloalkyl, and C₃-₈ heterocycloalkyl, wherein the heterocycloalkyl groups are optionally substituted with 1-4 R^{2c} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁-₆ alkyl;
each R^{2c} is independently selected from the group consisting of hydrogen, halogen, hydroxy, C₁-₆ alkoxy, C₁-₆ haloalkoxy, CN, and NR^{2a}R^{2b};
each R^{2d} is independently selected from the group consisting of hydrogen and C₁-₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁-₆ haloalkyl; and
subscript n is an integer from 0 to 3;
or salts and isomers thereof.

9. The method of claim 8, wherein the nonsteroidal SGRM is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, termed relacorilant, which has the following structure: 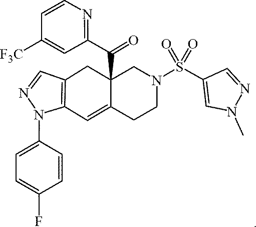
.

10. The method of claim 8, wherein the nonsteroidal SGRM is (R)-(1-(4-fluorophenyl)-6-((4-(trifluoromethyl)phenyl)sulfonyl)-4,4a,5,6,- 7,8-hexahydro-1H-pyrazolo[3,4-g]isoquinolin-4a-yl)(thiazol-2-yl)methanone, termed CORT122928, which has the following structure: 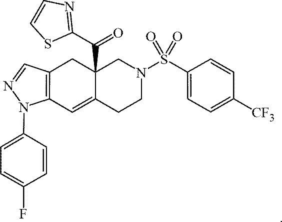
.

11. The method of claim 8, wherein the nonsteroidal SGRM is (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone, termed C0RT113176, which has the following structure: 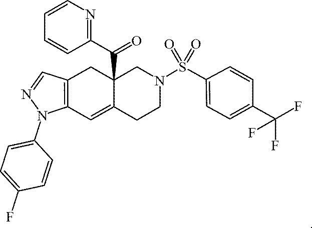
.

12. The method of claim 1, wherein the nonsteroidal SGRM comprises an octahydro fused azadecalin structure compound having the formula: 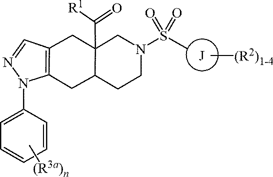
.
Wherein R¹ is selected from the group consisting of pyridine and thiazole, optionally substituted with 1-4 groups each independently selected from R^{1a};
each R^{1a} is independently selected from the group consisting of hydrogen, C₁-₆ alkyl, halogen, C₁-₆ haloalkyl, C₁-₆ alkoxy, C₁-₆ haloalkoxy, N-oxide, and C₃-₈ cycloalkyl; ring J is selected from the group consisting of phenyl, pyridine, pyrazole, and triazole;
each R² is independently selected from the group consisting of hydrogen, C₁-₆ alkyl, halogen, C₁-₆ haloalkyl, and —CN;
R^{3a} is F;
subscript n is an integer from 0 to 3,
or salts and isomers thereof.

13. The method of claim 12, wherein the nonsteroidal SGRM is ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone, termed exicorilant, which has the structure: 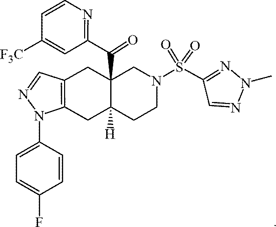
.

14. The method of claim 12, wherein the nonsteroidal SGRM is ((4aR,8aS)-1-(4-fluorophenyl)-6-((2-isopropyl-2H-1,2,3-triazol-4-yl)sulfonyl)-4,4a, 5,6,7,8,8a,9-octahydro-1H-pyrazolo[3,4-g]isoquinolin-4a-yl)(thiazol-2-yl)methanone, termed “CORT125329”, having the formula: 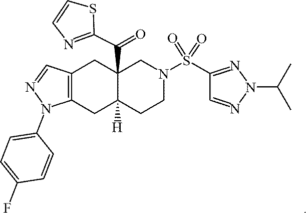
.

15. The method of claim 1, wherein the cancer treatment comprises administration of a chemotherapeutic agent.

16. The method of claim 15, wherein the chemotherapeutic agent is selected from the group consisting of taxanes, alkylating agents, topoisomerase inhibitors, endoplasmic reticulum stress inducing agents, antimetabolites, mitotic inhibitors and combinations thereof.

17. The method of claim 15, wherein the chemotherapeutic agent is a taxane.

18. The method of claim 17, wherein the chemotherapeutic agent is nabpaclitaxel.

19. The method of claim 1, wherein the cancer treatment comprises administration of an immunotherapeutic agent.

20. The method of claim 19, wherein the immunotherapeutic agent comprises administration of an antibody checkpoint inhibitor directed against a protein target selected from PD-1, PD-L1, CTLA-4, LAG3, B7-H3, B7-H4, OX-40, CD137, and TIM3.

21. The method of claim 1, wherein the cancer treatment comprises one or more of cancer radiation therapy, administration of growth factor inhibitors, and administration of anti-angiogenesis factors.

22. The method of claim 1, wherein said selective glucocorticoid receptor modulator is a selective glucocorticoid receptor antagonist.
